(19) United States
(12) Patent Application Publication
(10) Pub. No.: US 2003/0157575 A1
(43) Pub. Date: Aug. 21, 2003

(54) Chemical proteomics

(76) Inventors: Shane Climie, Toronto (CA); Leroi Victor DeSouza, North York (CA); Henry S. Duewel, Toronto (CA); Christopher Mark Hosfield, Toronto (CA); Linda Chiou-Lin Liao, Brampton (CA); Linda D. B. McBroom, Toronto (CA); Irving Sucholeiki, Winchester, MA (US); Leticia M. Toledo-Sherman, Venice, CA (US)

Correspondence Address: ROPES & GRAY LLP, ONE INTERNATIONAL PLACE, BOSTON, MA, 02110-2624, US

(21) Appl. No.: 10/352,517

(22) Filed: Jan. 28, 2003

Related U.S. Application Data
(60) Provisional application No. 60/352,458, filed on Jan. 28, 2002.
(60) Provisional application No. 60/427,743, filed on Nov. 20, 2002.

(51) Int. Cl. G01N 33/53; G01N 33/542

(52) U.S. Cl. 435/7.9

(57) ABSTRACT

The invention relates to methods and reagents for identifying/isolating protein targets of chemical compounds (for example, drug candidates) using mass spectrometry. The invention provides a method for capturing and identifying proteins using tethered small-molecule probes. This technology also allows the market expansion of known drugs by finding new therapeutic targets; identification of the mechanism of toxicity of drug candidates or drugs which failed in the clinic; identification of new chemical tools for chemically-driven target validation; identification of new drug leads; and identification of the mechanism of action of drugs and drug candidates. A key advantage of the technology is that a single experiment can identify the numerous proteins which interact with a probe (or “bait”).

### REFERENCE TO RELATED APPLICATIONS

This application claims priority to U.S. Provisional Applications No. 60/352,458, filed on Jan. 28, 2002 and Ser. No. 60/427,743, filed on Nov. 20, 2002, the entire contents of which are incorporated by reference herein.

### BACKGROUND OF THE INVENTION

The pharmaceutical industry today faces two fundamental challenges in its drug development process, namely the identification of appropriate protein targets for disease intervention (“validated targets”) and the identification of high quality drug candidates which act specifically on these targets (“validated leads”). These two challenges are of paramount importance in the design of successful medicines. A goal of each major pharmaceutical company is to produce 2 to 4 new chemical entities (NCEs) per year, but in reality the current output averages only 0.5 to 1 per year (Jain Report, 2001). The cost of drug development is estimated to be in the range of from about $400 to about $900 million. It is well established that a major factor in this expense is the failure to halt work on unsuccessful compounds early enough in the development process. This is no fault of the industry, as there is a dearth of tools available to aid in the decision-making process. Technologies which improve the drug development process will have significant impact on the industry.

It is clear that pharmaceutical companies do not lack targets; rather, they lack “validated” targets. With the recent completion of the Human Genome Project the potential number of target gene sequences available to the pharmaceutical industry has increased considerably. Given that a single gene can produce several protein variants, and that as many as 70% of proteins identified have no known function, a colossal task remains, namely that of drawing the link between the gene sequence of a potential target and a disease pathology appropriate for therapeutic intervention. This is not a straightforward task, but is aided by some of the tools emerging from the Proteomics industry.

The field of Proteomics applies specific methods and technologies to address fundamental questions about protein expression and function. Amongst other things, these technologies enumerate which proteins are expressed in both diseased and healthy tissues, the nature of how proteins interact with other cellular components, their localization patterns in the cell, their post-translational modification states when active and their specific involvement with signaling or metabolic pathways. Whereas the genome is a constant aspect of an organism, the proteome is dynamic, varying, for example, with the nature of the tissue, state of development, health or disease and effect of a drug. These features lead to a comprehensive molecular description and are key to providing a road map towards the discovery of new, more effective, medicines.

The use of chemical agents to study protein function and to identify protein targets has been at the heart of the emerging field of chemical genomics. Chemical agents which disrupt biological function have been used to find disease markers, validate targets and evaluate drug toxicity. These chemically-driven methods usually rely on mRNA levels as a readout of protein expression and activity. However, mRNA transcripts and expressed protein levels are only modestly correlated, if at all, and many regulatory processes occur after transcription. Chemical proteomics methods, which directly measure protein expression or function, are inherently more reliable than chemical genomics methods.

With recent developments in the field of proteomics, several so-called chemical proteomics techniques have appeared which use chemical probes to identify and isolate proteins from complex mixtures. These approaches can be categorized into affinity-based and activity-based Proteomics. Affinity-based methods, coupled to mass spectrometry, allow the identification of both synthetic and biological molecules. In one such approach a protein of interest (the “bait” protein) is immobilized on a solid support and proteins or small molecules which associate with the bait are identified by gel electrophoresis and mass spectrometry. In another approach poorly understood protein targets (immobilized, or as free proteins) are profiled against combinatorial libraries in search of small molecule ligands. Active ligands against the target can serve simultaneously as drug leads and modulators in chemically-driven target validation studies. However, these drug discovery or chemical genomics approaches are, in reality, protein-driven and require sources of already characterized and purified proteins, usually in relatively large amounts.

Activity-based chemical proteomics approaches permit the capture of proteins by taking advantage of the selective reactivity of a functional group involved in a protein's catalytic activity. The functional group in question is chemically-modified with reagents containing biotin tags, for example. In this way, “tagged” proteins can be separated from crude cell extracts by affinity chromatography and subsequently identified by Mass Spectrometry. For example, several members of a family of serine hydrolase enzymes were identified from a complex protein mixture using biotinylated flourophosphonate reagents (which specifically inhibit such enzymes). Recently the same group identified an aldehyde dehydrogenase using a biotinylated sulfonate ester library.

The two chemical proteomics methods described above are promising tools for discovering proteins of a given class and for identifying low abundance proteins, but suffer from a number of disadvantages. Activity-based methods do not query druggability or provide agents for target validation studies. Affinity-based chemoproteomics methods use as baits endogenous substrates, which are shared by many common proteins usually found in large numbers in cells (10% of all proteins make up 90% of the total protein mass of a cell). These proteins have to be fractionated by repetitive competitive elution in order to isolate the desired proteins. After fractionation, the isolated proteins are displaced by a soluble combinatorial library, in sequential fashion, and the binding affinity of individual compounds then estimated.

Further, due to the nature of the probes, neither of these methods is poised to discover the unknown; that is, serendipitous targets will not be found using these approaches. A general library of drug-like compounds used to capture any druggable target, or a gene-family specific library used to find new members of that family, would be a far more powerful tool.

Several companies have emerged which use micro-array technology to produce arrays of compounds for high throughput screening (HTS) against a single target. Whilst they use the term “chemical proteomics” to describe their work, these approaches do not contribute to the identification of new targets from complex proteomic mixtures and should instead be considered single target HTS methods rather than proteomics approaches.

### SUMMARY OF THE INVENTION

We have developed an approach for capturing and identifying proteins using small-molecule probes, which permits study of the direct effects of these molecules on protein levels and protein function. This approach uses resin-immobilized drug-like compound libraries as affinity probes to directly capture proteins from complex proteomes, coupled with Mass Spectrometry for the global analysis of protein expression levels in cells. For example, using this approach, cells treated with key drug-like compounds can be directly compared to untreated (or “control”) cells. The method disclosed herein uses structure-based drug design and computational chemistry techniques to design biologically- and/or structurally-relevant diverse drug-like chemical probes based upon pharmacophores known to modulate biological activities. The use of such a combiniatorial library allows the identification of proteins which are inherently “druggable.” This technology also allows the:

market expansion of known drugs by finding new therapeutic targets

identification of the mechanism of toxicity of drug candidates or drugs which failed in the clinic

identification of new chemical tools for chemically-driven target validation

identification of new drug leads

identification of the mechanism of action of drugs and drug candidates

A key advantage of the technology is that a single experiment can identify numerous proteins which interact with a probe (or “bait”).

Therefore, one aspect of the invention relates to a method of identifying protein target(s)-which interact with a chemical compound, comprising: (a) immobilizing said chemical compound on a support; (b) contacting said chemical compound immobilized on said support with a sample containing potential protein target(s); (c) isolating protein target(s) which interact with said immobilized chemical compound; (d) determining the identity of the protein target(s) isolated in (c) by mass spectrometry, thereby identifying protein target(s) of said chemical compound. In a preferred embodiment, said suport is a magnetic support. Any of the following embodiments or combination thereof, if applicable, may apply to this aspect of the invention.

In one embodiment, the sample is a cell lysate or a tissue extract. For example, said cell lysate can be from a primary human cell line or a tumor cell line. In a preferred embodiment, said cell lysate may be enriched for proteins specifically localized to a subcellular organelle (mitochondria, ER, neucleus, vacule, Golgi Complex, etc.) or a membrane faction (plasma membrane, nuclear membrane, etc.).

In one embodiment, said chemical compound has a desirable biological effect. In certain embodiments, the mechanism underlying said desirable biological effect may be unclear or incomplete. In certain embodiments, the method further comprises determining said mechanism by identifying one or more protein target(s) responsible for said desired biological effect. In certain embodiments, the method further comprises validating one or more identified protein target(s) of said chemical compound for a different desired biological effect.

In one embodiment, said chemical compound is a drug candidate having one or more undesirable side effect(s). In certain embodiments, the method further comprises determining the mechanism of said side effect(s) by identifying one or more protein target(s) responsible for said side effect(s). In certain embodiments, the method further comprises engineering said drug candidate to eliminate interaction with protein target(s) responsible for said side effect(s), without adversely affecting said desired biological effect(s).

In one embodiment, in step (a), the compound is synthesized on said magnetic support.

In one embodiment, said magnetic support is a polymeric solid support with desirable swelling properties in both organic and aqueous solvents.

In one embodiment, in step (a), said compound is immobilized on said magnetic support via a covalent linker. For example, said linker can be optimized for protein target interaction whilst minimizing undesirable nonspecific interactions. In certain embodiments, said linker is non-cleavable. In certain embodiments, said linker is photo-labile.

In one embodiment, in step (a), said compound is immobilized to said magnetic support via Biotin-Avidin affinity pair.

In one embodiment, said compound is Methotrexate (MTX).

In one embodiment, said magnetic support comprises a polyethylene glycol dimethylacrylamide (PEGA) copolymer.

In one embodiment, the mass spectrometry is tandem mass spectrometry.

In one embodiment, the mass spectrometry is Fourier Transform Mass Spectrometry (FTMS).

In one embodiment, said sample comprises a library of secondary samples, each independently obtained from a library of ADME/Tox assays. In a preferred embodiment, said secondary samples comprise a library of serum binding proteins.

Another aspect of the invention provides a method of optimizing interaction between a chemical compound and protein target(s) of said chemical compound, comprising: (a) providing a chemical compound having one or more desired biological effect(s); (b) identifying, by the method of claim 1, protein target(s) which interact with said chemical compound, wherein one or more of said protein target(s) has known structure; (c) designing, by computational chemistry methodology, a library of candidate chemical compounds derived from said chemical compound, taking into consideration the known structure of said target protein(s); (d) identifying, if any, one or more chemical compound(s) from the library of candidate chemical compounds, wherein said one or more chemical compound(s) each has an advantage when compared to said chemical compound, for example it interacts with said protein target(s) with higher affinity, or interacts with fewer targets, perhaps indicating higher specificity. In a preferred embodiment, step (b) is effectuated by the method of claim 2. Any of the following embodiments or combination thereof, when applicable, applies to this aspect of the invention.

In one embodiment, the method further comprises identifying and eliminating one or more undesirable chemical compounds which non-specifically interact with proteins from multiple pathways.

Another aspect of the invention provides a method of identifying interacting protein(s) for one or more compounds from a library of diverse chemical compounds having unknown biological activity, comprising: (a) providing said library of diverse chemical compounds by solid-phase synthesis which allows for cleavage of said chemical compounds from a support; (b) obtaining an equivalent portion of the library of chemical compounds in soluble form, for use in a panel of assays; (c) assessing selectivity of each member of the library of chemical compounds against the panel of assays; (d) identifying one or more compounds with selective efficacy in the panel of assays; (e) independently identifying, using the method of claim 1, protein target(s) of each of the one or more chemical compounds identified in (d). In a preferred embodiment, said support is a magnetic support, and wherein step (e) is effectuated by the method of claim 2. Any of the following embodiments or combination thereof, when applicable, applies to this aspect of the invention.

In one embodiment, step (b) is effected by cleavage of the library of chemical compounds from said magnetic support.

In one embodiment, said panel of assays relate to cellular assays which are disease models.

In one embodiment, step (e) is effected by directly using compounds synthesized in step (a).

In one embodiment, the panel of assays is a panel of ADME/Tox (Absorption, Distribution, Metabolism, and Excretion/Toxicity) assays.

In one embodiment, the panel of assays include assessing changes in expression level of proteins. In a preferred embodiment, the changes in expression level of proteins is assessed by FTMS (Fourier Transform Mass Spectrometry).

Another aspect of the invention provides a method of identifying new drug targets within a known protein target family, comprising: (a) providing a protein target family-specific, immobilized library of diverse chemical compounds based upon a chemical compound known to interact with said family, wherein said library of chemical compounds are immobilized on a support; (b) contacting said immobilized library of chemical compounds with a sample containing potential protein target(s); (c) isolating protein target(s) which interact with said immobilized library of chemical compounds; (d) determining the identity of, if any, new protein target(s) isolated in (c) by mass spectrometry, thereby identifying new drug target(s) within said known protein target family. In a preferred embodiment, said support is a magnetic support.

Another aspect of the invention provides a method of conducting a pharmaceutical business, comprising: (i) by the method of claim 1, identifying one or more interacting protein(s) of a chemical compound with known biological effects; (ii) validating the interacting protein(s) identified in step (i) as druggable disease targets, wherein the protein(s) were previously not known to be associated with diseases; (iii) formulating a pharmaceutical preparation including the chemical compounds for treatment of diseases associated with the protein target(s) identified in step (ii) as having an acceptable therapeutic profile. In a preferred embodiment, step (i) is effectuated by claim 2.

In one embodiment, the method includes an additional step of establishing a distribution system for distributing the pharmaceutical preparation for sale, and may optionally include establishing a sales group for marketing the pharmaceutical preparation.

Another aspect of the invention provides a method of conducting a pharmaceutical business, comprising: (i) by the method of claim 1, identifying one or more interacting protein(s) of a compound with known biological effects; (ii) licensing, to a third party, the rights for further drug development or target validation of the protein(s) identified in step (i). In a preferred embodiment, step (i) is effectuated by claim 2.

### BRIEF DESCRIPTION OF THE DRAWINGS

FIG. 1. A. Crystal structure of Methotrexate complexed within the active site of dihydrofolate reductase showing the γ-carboxylate protruding out of the cavity. B. Methotrexate molecule.

FIG. 2. Lane 1: Total lysate; 2: Marker; 3: Blank; 4: Eluate from column I; 5: Eluate from column 2; 6: Eluate from column 3; 7: Eluate from column 4; 8: Eluate from control column (column 5); 9: Eluate from column 6. Note: All columns were eluted w/ free MTX after washing with the corresponding buffer. Bands were excised from lanes 5, 7 and 9.

FIG. 3. Proteins denoted are a composite from results obtained from 3 lanes (i. e. lanes 5, 7 and 9 in FIG. 2). Enzymes also identified in the previous run are in normal text; Enzymes identified in this set of runs and whose connections to MTX are explained in this report are in bold text; Enzymes identified in this run but whose connection to MTX remains to be explained are in italic text.

FIG. 4. Affinity purification of HEI293 cell lysate with MTX-agarose. Lane 1. Molecular weight markers. Lane 2. Proteins eluted from MTX-agarose with 10 mM MTX.

FIG. 5. purine and pyrimidine de novo and salvage pathways showing enzymes isolated by the Methotrexate probe.

FIG. 6. Crystal structure of A. mtx-DHFR (1RG7), B. mtx-TS (1AXW), and C. folate-GART (1CDE), respectively showing γ-carboxylate of methotrexate or folate derivative protruding out of the binding cavities of all three enzymes.

FIG. 7. Overlap of docking poses (white) for methotrexate over the experimentally observed positions (gold) for all proteins. RMS (Å) deviations were A) 0.41 for mtx-DHFR (1RG7), B) 1.07 for mtx-TS-DUMP (1AXW), and C) 0.82 for folate-GART (1 CDE), respectively.

FIG. 8. Synthesis of L-methotrexate attached to photolinked PEGA magnetic beads

### DETAILED DESCRIPTION OF THE INVENTION

Definition

For convenience, certain terms employed in the specification, examples, and appended claims are collected here.

“ADME/Tox”: One of the needs of increasing importance in drug discovery is the ability to assay a potential drug compound for its pharmacological properties. To be an effective drug, a compound not only must be active against a target, but it needs also to possess the appropriate ADME (Absorption, Distribution, Metabolism, and Excretion) properties necessary to make it suitable for use as a drug. A potential drug should also be relatively non-toxic, or at least within a certain level of tolerable toxicity (Tox). For many years, much of this testing was done in vivo. However, with the increasing numbers of targets and hits being generated at most pharmaceutical companies, the need to do more ADME/Tox screening (particularly in vitro ADME testing) has become critical. A number of companies, such as Tecan Group Ltd. (Männedorf, Switzerland), offer commercial ADME/Tox assays. Other companies, such as Pharma Algorithms (Toronto, Canada) which develops software tools for molecular discovery in pharmaceutics and biotechnology, offer analysis means for ADME/Tox screen results using filters developed on basis of animal data. For example, its “Tox filter” is based on prediction of acute toxicity obtained from analysis of >30,000 compounds with LD₅₀ values in mouse (intraperitoneal administration). These and other equivalent commercial offerings can be used in the instant invention.

“Binding,” “bind”, “bound”, “immobilize”, “immobilized”, “tethered” or “tethering” refers to an association, which may be a stable association between two molecules, e.g., between a modified protein ligand an affinity capture reagent, due to, for example, electrostatic, hydrophobic, ionic and/or hydrogen-bond interactions under physiological conditions.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “Interacting Protein” is meant to include polypeptides that interact either directly or indirectly with another protein. Direct interaction means that the proteins may be isolated by virtue of their ability to bind to each other (e.g. by coimmunoprecipitation or other means). Indirect interaction refers to proteins which require another molecule in order to bind to each other. Alternatively, indirect interaction may refer to proteins which never directly bind to one another, but interact via an intermediary.

The term “isolated”, as used herein with reference to the subject proteins and protein complexes, refers to a preparation of protein or protein complex that is essentially free from contaminating proteins that normally would be present in association with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof It is to be understood, however, that such an “isolated” complex may incorporate other proteins the modulation of which, by the subject protein or protein complex, is being investigated.

“Analyzing a protein by mass spectrometry” or similar wording refers to using mass spectrometry to generate information which may be used to identify or aid in identifying a protein. Such information includes, for example, the mass or molecular weight of a protein, the amino acid sequence of a protein or protein fragment, a peptide map of a protein, and the purity or quantity of a protein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 95-99% by weight, and most preferably at least 99.8% by weight, of biological macromolecules of the same type present (but water, buffers, and other small molecules; especially molecules having a molecular weight of less than 5000, can be present). The term “pure” as used herein preferably has the same numerical limits as “purified” immediately above. “Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

“Sample” as used herein generally refers to a type of source or a state of a source, for example, a given cell type or tissue. The state of a source may be modified by certain treatments, such as by contacting the source with a chemical compound, before the source is used in the methods of the invention.

“Solid support” or “carrier,” used interchangeably, refers to a material which is an insoluble matrix, and may (optionally) have a rigid or semi-rigid surface. Such materials may take the form of small beads, pellets, disks, chips, dishes, multi-well plates, wafers or the like, although other forms may be used. In some embodiments, at least one surface of the substrate will be substantially flat.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 20% identity, though preferably less than 15% identity with a sequence of the present invention. Similarly, “homology” or “homologous” refers to sequences that are at least 20%, 25%, 30%, 35%, 40%, 45%, 50%, 55%, 60%, 65%, 70%, 75%, 80%, 85%, 90%, or even 95% to 99% identical to one another.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both polypeptide and DNA databases.

“Phospho-protein” is meant a polypeptide that can be potentially phosphorylated on at least one residue, which can be either tyrosine or serine or threonine or any combination of the three. Phosphorylation can occur constitutively or be induced.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

Overview

The revolution in combinatorial chemistries of the last decade has produced a large arsenal of diverse drug-like compounds, and the number of chemistries and chemotypes which are addressable by high throughput solid-support methodologies continues to grow. Many of these chemotypes have been found to be active against protein targets and target families of high interest to the pharmaceutical industry. Others have been reported to have interesting biological activity, but the exact molecular mechanism of action has not been identified. These compounds represent interesting entry points for probing proteome mixtures. They represent pharmacophore scaffolds which can be chemically modified to yield drug-like chemical probes, as single compounds or as combinatorial libraries.

In parallel with the developments in combinatorial chemistry, the field of structural biology has undergone a similar development over the last decade. The number of protein structures solved by X-ray crystallography and NMR methods has grown from a few thousand in the early 90's to over 110,000 today, with large numbers now being solved in high throughput fashion as part of publicly and privately funded initiatives. The collection of structures in protein databanks already contains a reasonable representation of domain folds (about 350 folds and 1,200 families). Many of these structures are of protein-ligand complexes; the identity of proteins and ligands can be correlated with the structure-based interests and activities of the pharmaceutical industry. Moreover, the bound ligands can be grouped into a few predominant categories: co-factors, substrates, compounds from medicinal chemistry efforts, or new compounds from the emerging arsenal of combinatorial drug-like entities. The majority of these ligands represent agonists or antagonists of the proteins and, as such, are potentially useful chemical probes. By nature, most binding sites have a solvent-exposed entrance, which allows for ligand binding. From a structural point of view any of these ligands can be used as starting point for the structure-based design of chemical probes expected to retain binding affinities to these proteins.

Computational chemistry applications allow for the structure-based design of compounds against targets whose structure is known, or which can be modeled from homologous proteins. These methods have been successfully applied to the design and understanding of important drugs such as HIV reverse-transcriptase inhibitor drugs. Methods based upon Quantitative Structure Activity Relationships (QSAR), on the other hand, allow correlations between the structure of a compound and a given biological activity. Such methods are used in the lead optimization process when the structure of the biological target is unknown. Typically, these can guide chemistry efforts by identifying regions of a molecule which can be chemically modified without losing the desired biological effect. Such computational chemistry methodologies can also be used in the design of compound probes.

The technology described in this application represents a tool to facilitate accurate selection of targets that are inherently druggable. By combining in-house proteomics technology with a chemical probe approach, disease-associated proteins can be identified directly. This permits a certain parallelism to the drug discovery process which is unprecedented. Such technology leads to fewer dropout compounds in the development pipeline and the rational drug design of compounds with fewer side effects.

One aspect of the invention employs a drug for which a mode of action is known, and structural and/or Structure Activity Relationship (SAR) information is understood, to design a probe to find new targets for therapeutic intervention and to explore the selectivity profile of such a compound against a given proteome. Then, using an appropriate chemical scaffold, a target-family specific diverse analog library can be designed in order to find new members of the given target family. In other words, scaffolds known to broadly inhibit a target family are identified, and then as diverse a library as possible is designed (to increase the diversity of the analog chemical space) in order to increase the odds of finding new members of the family. In the drug design process selectivity is often difficult to attain, especially in cases where inhibitors are directed to one member of a large gene family which shares structural homology. In the target-family directed probe approach described herein we take advantage of this very fact as a way to find new members. The use of resins loaded with target specific compound libraries allows the discovery of new druggable members of already fruitful drug discovery target families (e.g. kinases, proteases (caspases), phosphatases etc.).

The family of protein kinases can be used as illustration. It is estimated that the human genome encodes for over 500 members of this super family. This important class of proteins is at the heart of signal transduction pathways and has been implicated in many proliferative disorders such a cancer and psoriasis, disorders of the immune system, asthma and allergy, among others. Targets of this family are amenable to structure-based drug design methods which have already generated the post-genomic drug Gleevec, which has well-understood molecular mechanisms of action and few side effects. Approximately a dozen more kinase drugs are in different stages of pre-clinical and clinical development. However, the actual number of well-validated kinase targets is relatively small. Identifying new inherently druggable and disease-relevant proteins of this family, as new points of intervention, will have a significant impact in the industry. A library of general kinase inhibitors on a solid support can serve to identify new members of this already fruitful gene family.

A second aspect of the invention uses a library of diverse drug-like molecules having unknown biological activity to simultaneously look for important serendipitous targets and compound leads. This diverse library is assembled by solid-phase synthesis using methodology which allows for cleavage from the support. An equivalent portion of the library is available in, soluble form for cell assays. Such cellular assays for disease models include, but are not limited to, tumor cell proliferation, survival, and migration, cell responses to chemokines and cytokines (IL-1, TNF, IL-4, IL-10, IL-18, rantes, MCP-1, eotaxin, etc.), insulin-receptor mediated glucose metabolism and hormone signaling. Selectivity is assessed by profiling active compounds against the cellular activity panel. Compounds which show selective efficacy in these models (i.e. active in one model, but not generally cytotoxic) are then used as tethered baits to identify their molecular target from cell lysates, and to study the function of that target.

Such tethered small molecule baits are exposed to an appropriate cell lysate or tissue extract to identify novel target interactors. Mass Spectrometry can be used to study the effect of the equivalent soluble bait in cells. For example, valuable information on the differential expression of proteins in cells treated and non-treated with drug can thus be obtained. This allows the study of the effect of the drug directly on protein levels. In cases where the inhibitor inhibits a signaling cascade (kinases or phosphatases), phospho-profiling can be performed using proprietary methodology for the enrichment of phosphate-containing proteins.

Using this chemical proteomics technology, lead molecules, their molecular targets, mechanism(s) of action, selectivity and efficacy can be assessed at the same time, dramatically improving the drug discovery process and decreasing the attrition rate of compounds in clinical development pipelines.

One of the most expensive, yet important aspects in drug discovery and development is the clinical evaluation of emerging therapeutics; it is at this stage that most drug candidates are withdrawn, for example because they fail to show efficacy or have unacceptable side effects. One of the most promising aspects of the emerging field of Proteomics is the development of sensitive tools and methods which facilitate an understanding of the interactions between candidate drugs and their targets at the molecular level. Such information enables those compounds likely to fail in the clinic to be identified at the pre-clinical stage, such that only those compounds having more desirable properties will actually enter the clinic.

The use of drug-like tethered molecules as affinity probes to identify proteins directly from cell lysates or tissue samples offers the advantage of identifying proteins that are inherently druggable. There is a wealth of structural information and SAR on biologically relevant chemotypes amenable to solid phase synthesis. An important advantage of the approach disclosed herein is the seamless integration of synthetic and proteomics methodologies, as these compounds will be synthesized, purified and used to probe proteome mixtures directly on the solid support used for synthesis, without the need for chemical cleavage. This approach allows the fast assembly and efficient use of a large arsenal of chemical probes, and also facilitates the move from chemistry to protein identification. Through the design process a high measure of selectivity (or match) between bound protein and probe results. Thus, application of this technology to search for new members of a target family with an analog library results not only in the identification of new target members, but also in the identification of highly selective compounds for that target. The chemical entities used as probes represent drug leads against an identified protein and serve as tools for the investigation of protein function and validation.

Another aspect of the invention involves the use of the technology disclosed herein as a general drug discovery tool. This chemical proteomics approach facilitates the understanding of functional protein targets and provides tools for dissecting complex cellular processes. The use of compounds as modulators (with knowledge of the precise biological target(s)) to perturb the biological function of the targets contributes to target validation. Tethered molecules, as well as their resin-free counterparts, are useful molecular tools for accelerating target validation processes.

In the drug discovery process, knowledge of the specific pathways a compound activates allows specificity to be engineered-in and undesirable properties engineered-out earlier on the optimization process. Exact knowledge of the target(s) of a lead candidate helps direct chemical optimization towards producing a selective compound having a greater chance of success in the clinic.

Another aspect of the invention is the identification of novel indications for existing, approved drugs. For purposes of illustration consider a drug which is a kinase inhibitor. Given the large number of kinases expected to exist, is highly likely that this compound inhibits other opportunistic kinase targets involved in pathologies of broader impact. Therefore, it is reasonable to predict that the market potential of this compound could be greatly increased.

Another aspect of the invention is its use in defining the mechanism of action of an early drug candidate. In the scenario where a drug candidate exhibits an interesting biological effect, but for which the general molecular mechanism is unknown, the technology can be used to allow rational optimization of activity. For example, if a company has a small molecule lead or a class of molecules that exhibit an interesting biological effect and efficacy in a given disease model, but the exact mechanism of action is not understood, identification of effect-related targets will serve to facilitate their development into drugs. If structure-activity relationship data is available, regions of the molecule can be identified that can be modified without abolishing biological activity. Tethering this drug candidate allows proteomics analysis to identify the target(s) of the compound. Information of this sort is of tremendous value in the optimization process, especially when the target of interest is amenable to structure-based drug design.

Another aspect of the invention is its use in the “rescue” of drugs which failed in the clinic. For example, in the event that a drug failed in the clinic due to adverse side effects, the technology can be used to uncover the causative molecular mechanisms. Identifying all other pharmacodynamic targets inhibited by the drug would be of great value. This provides the information required to chemically modify the drug to tune out undesired side effects.

Another aspect of the invention is its use as a technique for ADME/Tox-profiling. The technology disclosed herein can be used to generate toxicity profiles and evaluate the ADME properties of drug candidates before they are introduced into the clinic. The pharmacokinetic properties of a drug candidate can be assessed by exposing the compound or compound class to a battery/panel of ADME/Tox relevant proteomes (i.e. serum binding proteins for use in, for example, assessing bio-availability of a potential drug), which provides important information useful in lead prioritization and lead optimization stages. Given several possible lead classes to take onto lead optimization, a quick assessment of the properties of each class helps the chemist select which class to focus on. The class most likely to have good ADME properties is most likely to generate a drug candidate that has the desired properties for drug development. Equally, knowledge of the secondary and tertiary targets for such compounds will reduce the occurrence of potentially toxic side effects, thus increasing the success rate in clinical development. In general, this technique can be used as a filter to prioritize which compounds to take into more rigorous and expensive pharmacokinetics and toxicology studies. ADME/Tox assays can be performed both in vivo and in vitro. Some companies (such as Tecan) offer commercial plateforms for performing such in vitro assays.

Another aspect of the invention is in the generation of chemical diagnostic markers. As an offshoot of the data generated from the use of the technology, it is possible to use the small molecule probes to identify protein markers for disease states. These can be developed into “chemical cards” in diagnostic kits, which can be used to monitor the status of a disease.

Another aspect of the invention is in the development of chemical micro arrayed chips. Miniaturized chips arrayed with compounds with drug-like properties (selected from specific libraries) can be used in high-throughput format as probes to identify druggable target proteins from a proteome of interest. This allows the parallel screening of a large number of compounds on a single chip and with several different proteomes (i.e. cell or tissue types).

Thus, the chemical proteomics platform described herein can be applied to solving fundamental problems and providing services to the pharmaceutical industry. The table below summarizes some of these, as well as the kinds of probes which can be used and the chemical ligand design strategy used. Practical details of the invention are discussed in the sections following.

| NATURE OF PROBE | PURPOSE | DESIGN STRATEGY |
|---|---|---|
| Target-family specific | To discover new | Design of a small |
| probe libraries | protein members of | focused library |
| | productive drug- | based on a chemotype |
| | discovery target | known to inhibit a |
| | families (e.g. | specific target family |
| | kinases, proteases, | using structure of |
| | ion channels, GPCRs, | target, homology |
| | phosphatases) | model or SAR (if |
| | To discover | available). |
| | compounds with | |
| | enhanced | |
| | selectivity profile | |
| | in a lead optimization | |
| | program against a | |
| | single or multiple | |
| | members of family. | |
| | To discover | |
| | compounds for tools in | |
| | chemical-driven target | |
| | validation studies. | |
| Diverse drug-like library | For the identi- | Design a small diverse |
| | fication of any | drug-like libraries |
| | druggable target. | using diversity tools |
| Chemical probe based on | To expand the market | Design of probes |
| a marketed drug of | potential of good drugs | based on the drug, |
| limited application | having a limited | using a tether which |
| | therapeutic window. | does not abrogate |
| | | activity. Use |
| | | applicable SBDD and |
| | | QSAR methods. |
| Chemical probe based on | To discover target(s) | Design small libraries |
| known biological activity | responsible for | incorporating |
| but unknown protein | biological activity | pharmacophores |
| target. | | known to elicit |
| | | biological activity |
| | | (possibly many such |
| | | libraries). |
| Chemical probe-based | To discover target(s) | Design probes based |
| drugs which failed in | responsible for the | on the drug, ensuring |
| the clinic due to | side effects in order | that design does not |
| adverse side-effects | to improve next | abrogate activity. |
| | generation drug | |

Ligand Design

Structure-based docking and library enumeration methods are used to design compound libraries against a particular target or target family of interest. A set of diverse drug-like compounds can also be prepared to address serendipitous druggable targets for pharmaceutical development. For compounds whose structure is available, account is taken of the regiochemical placement of the tethering to the solid support so that the biological activity is not abrogated. In cases where only SAR is available, QSAR methods are used to find the attachment point. In simplistic terms, in the optimization of a compound class, the position of the molecule that is used as an anchor for tailoring solubility and ADME properties lends itself to use as a tether for solid support.

By way of example, such a battery of compound baits includes specific target-directed baits, target family-directed library baits, biological activity-directed baits and a library containing diverse drug-like chemotypes. For directed baits, virtual screening methodology is used to rank compounds probes based on predicted affinity to a given target structure or homology model. Docking and consensus scoring is used to prioritize compound probes. In the case of the drug-like diverse probes, combinatorial library enumeration tools and chemical diversity algorithms are used to select sets of compounds which best represents a diverse drug-like chemical space.

Since this methodology can be used not only to find new targets, but also to find leads for drug discovery and target validation work, both free and tethered versions of the compounds of interest are needed. To discriminate between proteins which bind to the bait in a specific fashion vs. those which bind non-specifically, methodology for designing control compounds based on isosteric molecular structures which lack important binding elements (i.e. key hydrogen bonding features), and thus lack inhibitory activity, are employed. Such compounds are used for elution to compete off non-specific binding proteins.

Chemistry Solid Supports and Linkers

Chemistry—Over the last decade the promise of combinatorial chemistry to deliver drugs in short timeframes has fueled advances in supporting technologies like high-throughput solid- and solution-phase chemistry. Many techniques are available for constructing libraries for biological screening as single compounds, mixtures or as large libraries by split-pool methods. Solid support chemistry allows reactions to be driven to completion by use of excess reagents facilitating simplified chemical workups. Developments in scavenging resins allow for high throughput solution phase chemistry, as well. Already a large number of classical organic reactions have been adapted to combinatorial approaches, permitting the elaboration of complex molecular scaffolds. A large selection of polymeric support and linkers exists which allow for easy cleavage from solid supports by acid, base, photolysis, and fluoride based methods, for example. Using combinatorial approaches alone, around 1000 unique chemotypes have been reported, and most of these have disclosed biological activities.

A selection of target-specific compounds, such as compounds having broad activities against distinct gene families, diverse drug-like libraries, as well as compounds which elicit a biological response but whose molecular target is not known, can be prepared. Such compounds can be prepared using synthetic methodologies appropriate to the synthetic feasibility of the chemotypes, for example by solid-phase chemistry using a methodology which allows production of both solid-supported and solution counterparts for cell assays and protein expression/function analysis. In cases where the chemistry is not amenable to solid-phase methodology, compounds can be prepared in solution and coupled to the appropriate solid support.

Solid Supports—Together with large compound collections and chemistries, combinatorial chemistry has yielded a plethora of reagents and supports for solution and solid-support synthesis. Many polymeric solid-supports having desirable swelling properties in both organic and aqueous solvents (which lend themselves to both chemical and biological applications) are available. For example, high-swelling, polar, yet chemically inert PEG grafted resins such as Tentagels, POEPS and PEGA are simultaneously amenable to chemistries in organic solvents and to biological assays in aqueous solutions. Such resins swell in aqueous solvents, allowing permeation of biomolecules, and have been used in assays against crude cell extracts. The technique disclosed herein takes advantage of the flexibility and efficiency of solid supports which allow chemical synthesis, purification and direct probing of crude biological mixtures. Different types of resins can be utilized, in order to find optimal properties for the purpose at hand. The use of-magnetic beads (such as those disclosed in U.S. Pat. No. 5,858,534) is also demonstrated—such a support allows the simple mixing of cell extracts with beads containing tethered compounds. The use of a magnetic field to hold the beads allows for washing, decanting and isolating the resins without the need for column chromatography.

Linkers—For attaching compounds to the solid support several tethering systems can be used. For example, covalent linkers between compound and solid support can be employed, combinatorial techniques being used to optimize factors such as the linker type, rigidity and length optimal for protein binding, whilst minimizing unwanted nonspecific interactions. One category of covalent linkers is the non-cleavable type. In this case, elution from the affinity support or column with a soluble (free) version of the tethered compound is necessary to compete the desired protein off the solid support. Alternatively, stringent buffer conditions can be used to release the bound protein. Another tethering system involves the use of photo-labile linkers which allow for clean photo-cleavage of the compounds. In this manner, once the desired protein(s) has been captured, the probe-protein complex can be cleaved from the support and washed off the column or isolated, in the case of magnetic supports, without need for competitive elution with other agents. Several photo labile linkers are available that are easily cleavable using 354 nm irradiation and have been successfully applied to solid-phase synthesis with clean product release.

Another tethering system is the well-known Biotin-Avidin affinity pair. This is the single most exploited affinity sequestering and separating technique for biological applications. The system is based on immobilizing avidin, streptavidin or neutravidin on a solid support. A biotinylated bait molecule is mixed with a cell lysate. This mixture is then loaded on the avidin-based affinity column and washed to elute non-specific binding proteins. The desired protein can then be released by washing with several available reagents. This interacting system has been optimized to minimize nonspecific interactions between the immobilized avidin and proteins passing through the column. A substantial amount of work indicates that monomeric neutravidin can be used to minimize nonspecific interactions with common proteins. Furthermore many chemical reagents are readily available which allow the biotinylation of small molecules having specific functional groups.

Cell Assays and Detection of Biological Activity

Cellular assays can be used for compounds having known biological activity in order to validate that the compound chosen to model the library has the expected cellular effect. For example, an anti-cancer kinase inhibitor can be tested for its ability to block proliferation which is dependent upon kinase activity of the known target. Such cell assays will serve to ensure that the reported effect is attained using the test compound or library, and to verify the integrity of compounds and cell line before proteomics analysis with the tethered library. In cases where a molecular target of the compound is known, then direct enzymatic assays and in vitro binding studies can be used to further probe the molecule and the associated biology. Enzymatic assays can be performed using both the original soluble compound as well as the compound on solid support; the latter study providing evidence that the attachment of the linker is not detrimental to protein binding.

Once all the above points have been confirmed, cells are lysed and exposed to the tethered small molecule baits to identify novel target interactors from the lysate. For example, in the kinase case study, since the initial compound probes are known kinase inhibitors, most of the targets identified will be kinases as well. Even the most advanced kinase inhibitors in clinical trials have only been tested against a small select number of the more than 500 predicted kinases. None of these compounds are truly specific, suggesting that they are likely to bind additional novel kinases when the entire proteome is probed. This information is valuable in the drug discovery process in the search and selection of second-generation kinase inhibitors.

Biological Sample Preparation, Proteome Probing and Separation

Sample Preparation: Protein interactors sequestered by the chemical bait can be identified from primary human cell lines. Such cell lines include HBEK 293 cells as a model cell line, in addition to cell lines having unique phenotypes for more comprehensive investigations. Again, using the kinase inhibitors as an example, tumor cell lines which express kinase oncogenes can be employed. Standard protocols are used to culture the various human cell lines. Cells maintained as suspension cultures are harvested by centrifugation, washed to remove culture media, and then suspended in one of two generic lysis buffer types. One buffer type is used when cells are mechanically or physically disrupted (e.g. homogenization) post-suspension; the other buffer type contain additives (e.g. detergents) to bring about cellular lysis and is used either for cells harvested from suspension cultures or for adherent cells grown on culture plates. Confluent adherent cells are washed prior to the addition of the lysis buffer and scraped to concurrently dislodge and lyse the cells using established methods. When required, a cocktail of protease inhibitors or an agonist of choice can be added to the lysis buffer. The strength of the lysis buffer is tailored to favor both protein-chemical bait and protein-protein interactions. Likewise, if membrane fractions or subcellular organelles are to be targeted, the composition of the lysis buffer can be adjusted to favor their isolation through differential centrifugation. Membrane fractions can require additional treatment with detergents in order to solubilize membrane proteins.

Affinity Purification: Once the lysate has been prepared and separated into the targeted cellular fraction (e.g. cytosolic, membrane, organelle), the fraction is probed with the chemical bait in either a batch or column format. In the batch format, the chemical bait bearing resin is added to the lysate fraction and then gently agitated. After a set incubation time, the resin is collected by centrifugation or filtration and washed to remove non-specific interactions to the resin backbone. In the column format, the resin is packed into a micro-column and the lysate fraction is subjected to affinity chromatography. Protein(s) and their binding partners specifically interacting with the tethered chemical bait are eluted through competition with a soluble chemical bait or with stringent buffers (e.g. high salt, extreme pH).

In cases in which the bait is tethered via a photo-labile linker, the resin is irradiated to cleave the bait and its associated proteins from the resin. The use of photo linkers is particularly attractive in conjunction with magnetic beads for the application of this technology to chemical micro-arrays. For example, split-pool synthesis of compound libraries attached to a magnetic solid-support can be arrayed on a magnetized surface. Individual beads containing compounds are then exposed to cell lysates and washed to eliminate unwanted interactions. Photolysis releases the ligand complexed with interacting proteins from the resin for MS analysis. Such an approach can be adopted as a microfluidic system for process parallelization.

Mass Spectrometry Analysis and Identification

Protein Analysis. Proteins eluted from the tethered bait can be separated by SDS-PAGE and detected by colloidal Coomassie or silver staining, and protein bands of interest excised and digested in-gel with trypsin. Alternatively, proteins eluted from the tethered bait can be digested with trypsin directly in solution. Proteins can be identified through combined analysis of the tryptic peptides by mass spectrometry and protein/DNA database searching using MDS Proteomic's in-house proteomics, mass spectrometry and bioinformatics tools.

MS Mechanism of Action and Pathway Analysis.

Once a drug target has been identified, study of the differential expression of proteins in a cell which has been treated with a drug vs. a (non-treated) control can be carried out, for example using Mass Spectrometry (MS). This allows the study of the effect of the drug directly on protein levels. In the event that the compound inhibits a signaling cascade (inhibitors of kinases or phosphatases) phospho-profiling can be carried out (using proprietary methodology, for example, for the enrichment of phosphate-containing proteins). Such an analysis allows the dissection of the various cellular pathways affected by the drug and, simultaneously, gains an understanding of protein function. This is particularly important in assessing drug efficacy in a disease model.

In a preferred embodiment, Fourier Transform Mass Spectrometry (FTMS), which offers several advantages over traditional electron multiplier-based mass spectroscopy, is used. FTMS combines desirable aspects of other instruments (resolution and mass accuracy) with improvements in detection limits and dynamic ranges. FTMS instruments currently being developed have detection limits 1-3 orders of magnitude better than any other MS instrument, single scan dynamic ranges of 1000-10,000 (1-2 orders of magnitude better), resolution of >10 k, and mass accuracy in the low pip range. These improvements in MS design allow more complex mixtures to be analyzed, giving rise to smaller sample handling losses, less sample requirements (because of the improved detection limits) and more confidence can be given to the results due to the resolution and mass accuracy advantages. In short, FTMS offers many new features and expands on the information which can be realized from an experiment.

Small-Molecule Micro-Array Coupled to Mass Spectrometry

Micro-array technology offers the possibility of multiplexing the discovery of small-molecule protein interactions. The construction of small molecule micro-arrays has been recently achieved. The application of such small molecule micro-arrays to date has been limited to the discovery of specific protein-small molecule interaction using highly purified proteins. The full power of micro-array technology can only be achieved once complex protein mixtures can be simultaneously screened by the micro-array.

The technology disclosed herein allows, for the first time, an approach which combines small-molecule micro-array with high-throughput mass spectrometry for the screening of complex protein mixtures. Micro-arrays using small molecule drug-like libraries that encode pharmacophoric features known to elicit a biological response can be developed. These micro-arrays can be used to screen cell lysates from cell culture and tissues. The proteins present in the lysate form specific interactions with the different small molecules immobilized on the array. Elements on the array are able to extract proteins from the lysate either by forming binary interactions or by pulling down protein complexes.

Clearly, the multiplicities of proteins which can be extracted by every element on the micro-array requires a detection technique which can unambiguously perform protein identification. Mass spectrometry, performed on the peptides obtained by proteolytic digestion of proteins present on the individual element of the array, provides unambiguous identification of the proteins. Multiple proteins can be extracted by every small-molecule element present on the array. Tandem mass spectrometry coupled with protein/DNA databases searching can identify the protein absorbed on the array. This technique is a valuable tool in finding diagnostic disease markers and targets for therapeutic intervention.

Mass Spectrometers, Detection Methods and Sequence Analysis

In certain embodiments, the isolated proteins are subjected to protease digestion followed by mass spectrometry. During the past decade, new techniques in mass spectrometry have made it possible to accurately measure with high sensitivity the molecular weight of peptides and intact proteins. These techniques have made it much easier to obtain accurate peptide masses of a protein for use in databases searches. Mass spectrometry provides a method, of protein identification that is both very sensitive (10 fmol -1 pmol) and very rapid when used in conjunction with sequence databases. Advances in protein and DNA sequencing technology are resulting in an exponential increase in the number of protein sequences available in databases. As the size of DNA and protein sequence databases grows, protein identification by correlative peptide mass matching has become an increasingly powerful method to identify and characterize proteins.

Mass Spectrometry

Mass spectrometry, also called mass spectroscopy, is an instrumental approach that allows for the gas phase generation of ions as well as their separation and detection. The five basic parts of any mass spectrometer include: a vacuum system; a sample introduction device; an ionization source; a mass analyzer; and an ion detector. A mass spectrometer determines the molecular weight of chemical compounds by ionizing, separating, and measuring molecular ions according to their mass-to-charge ratio (m/z). The ions are generated in the ionization source by inducing either the loss or the gain of a charge (e.g. electron ejection, protonation, or deprotonation). Once the ions are formed in the gas phase they can be electrostatically directed into a mass analyzer, separated according to mass and finally detected. The result of ionization, ion separation, and detection is a mass spectrum that can provide molecular weight or even structural information.

A common requirement of all mass spectrometers is a vacuum. A vacuum is necessary to permit ions to reach the detector without colliding with other gaseous molecules. Such collisions would reduce the resolution and sensitivity of the instrument by increasing the kinetic energy distribution of the ion's inducing fragmentation, or preventing the ions from reaching the detector. In general, maintaining a high vacuum is crucial to obtaining high quality spectra.

The sample inlet is the interface between the sample and the mass spectrometer. One approach to introducing sample is by placing a sample on a probe which is then inserted, usually through a vacuum lock, into the ionization region of the mass spectrometer. The sample can then be heated to facilitate thermal desorption or undergo any number of high-energy desorption processes used to achieve vaporization and ionization.

Capillary infusion is often used in sample introduction because it can efficiently introduce small quantities of a sample into a mass spectrometer without destroying the vacuum. Capillary columns are routinely used to interface the ionization source of a mass spectrometer with other separation techniques including gas chromatography (GC) and liquid chromatography (LC). Gas chromatography and liquid chromatography can serve to separate a solution into its different components prior to mass analysis. Prior to the 1980's, interfacing liquid chromatography with the available ionization techniques was unsuitable because of the low sample concentrations and relatively high flow rates of liquid chromatography. However, new ionization techniques such as electrospray were developed that now allow LC/MS to be routinely performed. One variation of the technique is that high performance liquid chromatography (HPLC) can now be directly coupled to mass spectrometer for integrated sample separation/preparation and mass spectrometer analysis.

In terms of sample ionization, two of the most recent techniques developed in the mid 1980's have had a significant impact on the capabilities of Mass Spectrometry: Electrospray Ionization (ESI) and Matrix Assisted Laser Desorption/Ionization (MALDI). ESI is the production of highly charged droplets which are treated with dry gas or heat to facilitate evaporation leaving the ions in the gas phase. MALDI uses a laser to desorb sample molecules from a solid or liquid matrix containing a highly UV-absorbing substance.

The MALDI-MS technique is based on the discovery in the late 1980s that an analyte consisting of, for example, large nonvolatile molecules such as proteins, embedded in a solid or crystalline “matrix” of laser light-absorbing molecules can be desorbed by laser irradiation and ionized from the solid phase into the gaseous or vapor phase, and accelerated as intact molecular ions towards a detector of a mass spectrometer. The “matrix” is typically a small organic acid mixed in solution with the analyte in a 10,000:1 molar ratio of matrix/analyte. The matrix solution can be adjusted to neutral pH before mixing with the analyte.

The MALDI ionization surface may be composed of an inert material or else modified to actively capture an analyte. For example, an analyte binding partner may be bound to the surface to selectively absorb a target analyte or the surface may be coated with a thin nitrocellulose film for nonselective binding to the analyte. The surface may also be used as a reaction zone upon which the analyte is chemically modified, e.g., CNBr degradation of protein. See Bai et al, Anal. Chem. 67, 1705-1710 (1995).

Metals such as gold, copper and stainless steel are typically used to form MALDI ionization surfaces. However, other commercially-available inert materials (e.g., glass, silica, nylon and other synthetic polymers, agarose and other carbohydrate polymers, and plastics) can be used where it is desired to use the surface as a capture region or reaction zone. The use of Nation and nitrocellulose-coated MALDI probes for on-probe purification of PCR-amplified gene sequences is described by Liu et al., Rapid Commun. Mass Spec. 9:735-743 (1995). Tang et al. have reported the attachment of purified oligonucleotides to beads, the tethering of beads to a probe element, and the use of this technique to capture a complimentary DNA sequence for analysis by MALDI-TOF MS (reported by K. Tang et al., at the May 1995 TOF-MS workshop, R. J. Cotter (Chairperson); K. Tang et al., Nucleic Acids Res. 23, 3126-3131, 1995). Alternatively, the MALDI surface may be electrically- or magnetically activated to capture charged analytes and analytes anchored to magnetic beads respectively.

Aside from MALDI, Electrospray Ionization Mass Spectrometry (ESI/MS) has been recognized as a significant tool used in the study of proteins, protein complexes and bio-molecules in general. ESI is a method of sample introduction for mass spectrometric analysis whereby ions are formed at atmospheric pressure and then introduced into a mass spectrometer using a special interface. Large organic molecules, of molecular weight over 10,000 Daltons, may be analyzed in a quadrupole mass spectrometer using ESI.

In ESI, a sample solution containing molecules of interest and a solvent is pumped into an electrospray chamber through a fine needle. An electrical potential of several kilovolts may be applied to the needle for generating a fine spray of charged droplets. The droplets may be sprayed at atmospheric pressure into a chamber containing a heated gas to vaporize the solvent. Alternatively, the needle may extend into an evacuated chamber, and the sprayed droplets are then heated in the evacuated chamber. The fine spray of highly charged droplets releases molecular ions as the droplets vaporize at atmospheric pressure. In either case, ions are focused into a beam, which is accelerated by an electric field, and then analyzed in a mass spectrometer.

Because electrospray ionization occurs directly from solution at atmospheric pressure, the ions formed in this process tend to be strongly solvated. To carry out meaningful mass measurements, solvent molecules attached to the ions should be efficiently removed, that is, the molecules of interest should be “desolvated.” Desolvation can, for example, be achieved by interacting the droplets and solvated ions with a strong countercurrent flow (6-9 l/m) of a heated gas before the ions enter into the vacuum of the mass analyzer.

Other well-known ionization methods may also be used. For example, electron ionization (also known as electron bombardment and electron impact), atmospheric pressure chemical ionization (APCI), fast atom Bombardment (FAB), or chemical ionization (CI).

Immediately following ionization, gas phase ions enter a region of the mass spectrometer known as the mass analyzer. The mass analyzer is used to separate ions within a selected range of mass to charge ratios. This is an important part of the instrument because it plays a large role in the instrument's accuracy and mass range. Ions are typically separated by magnetic fields, electric fields, and/or measurement of the time an ion takes to travel a fixed distance.

If all ions with the same charge enter a magnetic field with identical kinetic energies a definite velocity will be associated with each mass and the radius will depend on the mass. Thus a magnetic field can be used to separate a monoenergetic ion beam into its various mass components. Magnetic fields will also cause ions to form fragment ions. If there is no kinetic energy of separation of the fragments the two fragments will continue along the direction of motion with unchanged velocity. Generally, some kinetic energy is lost during the fragmentation process creating non-integer mass peak signals which can be easily identified. Thus, the action of the magnetic field on fragmented ions can be used to give information on the individual fragmentation processes taking place in the mass spectrometer.

Electrostatic fields exert radial forces on ions attracting them towards a common center. The radius of an ion's trajectory will be proportional to the ion's kinetic energy as it travels through the electrostatic field. Thus an electric field can be used to separate ions by selecting for ions that travel within a specific range of radii which is based on the kinetic energy and is also proportion to the mass of each ion.

Quadrupole mass analyzers have been used in conjunction with electron ionization sources since the 1950s. Quadrupoles are four precisely parallel rods with a direct current (DC) voltage and a superimposed radio-frequency (RF) potential. The field on the quadrupoles determines which ions are allowed to reach the detector. The quadrupoles thus function as a mass filter. As the field is imposed, ions moving into this field region will oscillate depending on their mass-to-charge ratio and, depending on the radio frequency field, only ions of a particular m/z can pass through the filter. The m/z of an ion is therefore determined by correlating the field applied to the quadrupoles with the ion reaching the detector. A mass spectrum can be obtained by scanning the RF field. Only ions of a particular m/z are allowed to pass through.

Electron ionization coupled with quadrupole mass analyzers can be employed in practicing the instant invention. Quadrupole mass analyzers have found new utility in their capacity to interface with electrospray ionization. This interface has three primary advantages. First, quadrupoles are tolerant of relatively poor vacuums (˜5×10⁻⁵ torr), which makes it well-suited to electrospray ionization since the ions are produced under atmospheric pressure conditions. Secondly, quadrupoles are now capable of routinely analyzing up to an m/z of 3000, which is useful because electrospray ionization of proteins and other biomolecules commonly produces a charge distribution below m/z 3000. Finally, the relatively low cost of quadrupole mass spectrometers makes them attractive as electrospray analyzers.

The ion trap mass analyzer was conceived of at the same time as the quadrupole mass analyzer. The physics behind both of these analyzers is very similar. In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consist of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement.

Quadrupole ion traps can be used in conjunction with electrospray ionization MS/MS experiments in the instant invention.

The earliest mass analyzers separated ions with a magnetic field. In magnetic analysis, the ions are accelerated (using an electric field) and are passed into a magnetic field. A charged particle traveling at high speed passing through a magnetic field will experience a force, and travel in a circular motion with a radius depending upon the m/z and speed of the ion. A magnetic analyzer separates ions according to their radii of curvature, and therefore only ions of a given m/z will be able to reach a point detector at any given magnetic field. A primary limitation of typical magnetic analyzers is their relatively low resolution.

In order to improve resolution, single-sector magnetic instruments have been replaced with double-sector instruments by combining the magnetic mass analyzer with an electrostatic analyzer. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2 V/E allows one to determine that only ions of energy V will be allowed to pass. Thus, the addition of an electric sector allows only ions of uniform kinetic energy to reach the detector, thereby increasing the resolution of the two sector instrument to 100,000. Magnetic double-focusing instrumentation is commonly used with FAB and EI ionization, however they are not widely used for electrospray and MALDI ionization sources primarily because of the much higher cost of these instruments. But in theory, they can be employed to practice the instant invention.

ESI and MALDI-MS commonly use quadrupole and time-of-flight mass analyzers, respectively. The limited resolution offered by time-of-flight mass analyzers, combined with adduct formation observed with MALDI-MS, results in accuracy on the order of 0.1% to a high of 0.01%, while ESI typically has an accuracy on the order of 0.01%. Both ESI and MALDI are now being coupled to higher resolution mass analyzers such as the ultrahigh resolution (>10⁵) mass analyzer. The result of increasing the resolving power of ESI and MALDI mass spectrometers is an increase in accuracy for biopolymer analysis.

Fourier-transform ion cyclotron resonance (FTMS) offers two distinct advantages, high resolution and the ability to tandem mass spectrometry experiments. FTMS is based on the principle of a charged particle orbiting in the presence of a magnetic field. While the ions are orbiting, a radio frequency (RF) signal is used to excite them and as a result of this RRF excitation, the ions produce a detectable image current. The time-dependent image current can then be Fourier transformed to obtain the component frequencies of the different ions which correspond to their m/z.

Coupled to ESI and MALDI, FTMS offers high accuracy with errors as low as ±0.001%. The ability to distinguish individual isotopes of a protein of mass 29,000 is demonstrated.

A time-of-flight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determine from the ions' time of arrival.

The arrival time of an ion at the detector is dependent upon the mass, charge, and kinetic energy of the ion. Since kinetic energy (KE) is equal to ½ mv² or velocity v=(2 KE/m)^{1/2}, ions will travel a given distance, d, within a time, t, where t is dependent upon their m/z.

The magnetic double-focusing mass analyzer has two distinct parts, a magnetic sector and an electrostatic sector. The magnet serves to separate ions according to their mass-to-charge ratio since a moving charge passing through a magnetic field will experience a force, and travel in a circular motion with a radius of curvature depending upon the m/z of the ion. A magnetic analyzer separates ions according to their radii of curvature, and therefore only ions of a given m/z will be able to reach a point detector at any given magnetic field. A primary limitation of typical magnetic analyzers is their relatively low resolution. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2 V/E allows one to determine that only ions of energy V will be allowed to pass. Thus, the addition of an electric sector allows only ions of uniform kinetic energy to reach the detector, thereby increasing the resolution of the two sector instrument.

The new ionization techniques are relatively gentle and do not produce a significant amount of fragment ions, this is in contrast to electron ionization (EI) which produces many fragment ions. To generate more information on the molecular ions generated in the ESI and MALDT ionization sources, it has been necessary to apply techniques such as tandem mass spectrometry (MS/MS), to induce fragmentation. Tandem mass spectrometry (abbreviated MSn—where n refers to the number of generations of fragment ions being analyzed) allows one to induce fragmentation and mass analyze the fragment ions. This is accomplished by collisionally generating fragments from a particular ion and then mass analyzing the fragment ions.

Tandem mass spectrometry or post source decay is used for proteins that cannot be identified by peptide-mass matching or to confirm the identity of proteins that are tentatively identified by an error-tolerant peptide mass search, described above. This method combines two consecutive stages of mass analysis to detect secondary fragment ions that are formed from a particular precursor ion. The first stage serves to isolate a particular ion of a particular peptide (polypeptide) of interest based on its m/z. The second stage is used to analyze the product ions formed by spontaneous or induced fragmentation of the selected ion precursor. Interpretation of the resulting spectrum provides limited sequence information for the peptide of interest. However, it is faster to use the masses of the observed peptide fragment ions to search an appropriate protein sequence database and identify the protein as described in Griffin et al, Rapid Commun. Mass. Spectrom. 1995, 9: 1546. Peptide fragment ions are produced primarily by breakage of the amide bonds that join adjacent amino acids. The fragmentation of peptides in mass spectrometry has been well described (Falick et al., J. Am Soc. Mass Spectrom. 1993, 4, 882-893; Bieniann, K., Biomed. Environ. Mass Spectrom. 1988, 16, 99-111).

For example, fragmentation can be achieved by inducing ion/molecule collisions by a process known as collision-induced dissociation (CID) or also known as collision-activated dissociation (CAD). CID is accomplished by selecting an ion of interest with a mass filter/analyzer and introducing that ion into a collision cell. A collision gas (typically Ar, although other noble gases can also be used) is introduced into the collision cell, where the selected ion collides with the argon atoms, resulting in fragmentation. The fragments can-then be analyzed to obtain a fragment ion spectrum. The abbreviation MSn is applied to processes which analyze beyond the initial fragment ions (MS2) to second (MS3) and third generation fragment ions (MS4). Tandem mass analysis is primarily used to obtain structural information, such as protein or polypeptide sequence, in the instant invention.

In certain instruments, such as those by JEOL USA, Inc. (Peabody, Mass.), the magnetic and electric sectors in any JEOL magnetic sector mass spectrometer can be scanned together in “linked scans” that provide powerful MS/MS capabilities without requiring additional mass analyzers. Linked scans can be used to obtain product-ion mass spectra, precursor-ion mass spectra, and constant neutral-loss mass spectra. These can provide structural information and selectivity even in the presence of chemical interferences. Constant neutral loss spectrum essentially “lifts out” only the interested peaks away from all the background peaks, hence removing the need for class separation and purification. Neutral loss spectrum can be routinely generated by a number of commercial mass spectrometer instruments (such as the one used in the Example section). JEOL mass spectrometers can also perform fast linked scans for GC/MS/MS and LC/MS/MS experiments.

Once the ion passes through the mass analyzer it is then detected by the ion detector, the final element of the mass spectrometer. The detector allows a mass spectrometer to generate a signal (current) from incident ions, by generating secondary electrons, which are further amplified. Alternatively some detectors operate by inducing a current generated by a moving charge. Among the detectors described, the electron multiplier and scintillation counter are probably the most commonly used and convert the kinetic energy of incident ions into a cascade of secondary electrons. Ion detection can typically employ Faraday Cup, Electron Multiplier, Photomultiplier Conversion Dynode (Scintillation Counting or Daly Detector), High-Energy Dynode Detector (HED), Array Detector, or Charge (or Inductive) Detector.

The introduction of computers for MS work entirely altered the manner in which mass spectrometry was performed. Once computers were interfaced with mass spectrometers it was possible to rapidly perform and save analyses. The introduction of faster processors and larger storage capacities has helped launch a new era in mass spectrometry. Automation is now possible allowing for thousands of samples to be analyzed in a single day. The use of computer also helps to develop mass spectra databases which can be used to store experimental results. Software packages not only helped to make the mass spectrometer more user friendly but also greatly expanded the instrument's capabilities.

The ability to analyze complex mixtures has made MALDI and ESI very useful for the examination of proteolytic digests, an application otherwise known as protein mass mapping. Through the application of sequence specific proteases, protein mass mapping allows for the identification of protein primary structure. Performing mass analysis on the resulting proteolytic fragments thus yields information on fragment masses with accuracy approaching ±5 ppm; or ±0.005 Da for a 1,000 Da peptide. The protease fragmentation pattern is then compared with the patterns predicted for all proteins within a database and matches are statistically evaluated. Since the occurrence of Arg and Lys residues in proteins is statistically high, trypsin cleavage (specific for Arg and Lys) generally produces a large number of fragments which in turn offer a reasonable probability for unambiguously identifying the target protein.

The primary tools in these protein identification experiments are mass spectrometry, proteases, and computer-facilitated data analysis. As a result of generating intact ions, the molecular weight information on the peptides/proteins are quite unambiguous. Sequence specific enzymes can then provide protein fragments that can be associated with proteins within a database by correlating observed and predicted fragment masses. The success of this strategy, however, relies on the existence of the protein sequence within the database. With the availability of the human genome sequence (which indirectly contain the sequence information of all the proteins in the human body) and genome sequences of other organisms (mouse, rat, Drosophila, C. elegans, bacteria, yeasts, etc.), identification of the proteins can be quickly determined simply by measuring the mass of proteolytic fragments.

Representative mass spectrometry instruments useful for practicing the instant invention are described in detail in the Examples. A skilled artisan should readily understand that other similar instruments with equivalent function/specification, either commercially available or user modified, are suitable for practicing the instant invention.

Protease Digestion

Prior to analysis by mass spectrometry, the protein may be chemically or enzymatically digested. For protein bands from gels, the protein sample in the gel slice may be subjected to in-gel digestion. (see Shevchenko A. et al., Mass Spectrometric Sequencing of Proteins from Silver Stained Polyacrylamide Gels. Analytical Chemistry 1996, 58: 850).

One aspect of the instant invention is that peptide fragments ending with lysine or arginine residues can be used for sequencing with tandem mass spectrometry. While trypsin is the preferred the protease, many different enzymes can be used to perform the digestion to generate peptide fragments ending with Lys or Arg residues. For instance, in page 886 of a 1979 publication of Enzymes (Dixon, M. et al. ed., 3rd edition, Academic Press, New York and San Francisco, the content of which is incorporated herein by reference), a host of enzymes are listed which all have preferential cleavage sites of either Arg- or Lys- or both, including Trypsin [EC 3.4.21.4], Thrombin [EC 3.4.21.5], Plasmin [EC 3.4.21.7], Kallikrein [EC 3.4.21.8], Acrosin [EC 3.4.21.10], and Coagulation factor Xa [EC 3.4.21.6]. Particularly, Acrosin is the Trypsin-like enzyme of spermatoza, and it is not inhibited by α1-antitrypsin. Plasmin is cited to have higher selectivity than Trypsin, while Thrombin is said to be even more selective. However, this list of enzymes are for illustration purpose only and is not intended to be limiting in any way. Other enzymes known to reliably and predictably perform digestions to generate the polypeptide fragments as described in the instant invention are also within the scope of the invention.

BLAST Search

The raw data of mass spectrometry will be compared to public, private or commercial databases to determine the identity of polypeptides.

BLAST search can be performed at the NCBI's (National Center for Biotechnology Information) BLAST website. According to the NCBI BLAST website, BLAST® (Basic Local Alignment Search Tool) is a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real, matches easier to distinguish from random background hits. BLAST uses a heuristic algorithm which seeks local as opposed to global alignments and is therefore able to detect relationships among sequences which share only isolated regions of similarity (Altschul et al., 1990, J. Mol. Biol. 215: 403-10). The BLAST website also offer a “BLAST course,” which explains the basics of the BLAST algorithm, for a better understanding of BLAST.

For protein sequence search, several protein-protein BLAST can be used. Protein BLAST allows one to input protein sequences and compare these against other protein sequences.

“Standard protein-protein BLAST” takes protein sequences in FASTA format, GenBank Accession numbers or GI numbers and compares them against the NCBI protein databases (see below).

“PSI-BLAST” (Position Specific Iterated BLAST) uses an iterative search in which sequences found in one round of searching are used to build a score model for the next round of searching. Highly conserved positions receive high scores and weakly conserved positions receive scores near zero. The profile is used to perform a second (etc.) BLAST search and the results of each “iteration” used to refine the profile. This iterative searching strategy results in increased sensitivity.

“PHI-BLAST” (Pattern Hit Initiated BLAST) combines matching of regular expression pattern with a Position Specific iterative protein search. PHI-BLAST can locate other protein sequences which both contain the regular expression pattern and are homologous to a query protein sequence.

“Search for short, nearly exact sequences” is an option similar to the standard protein-protein BLAST with the parameters set automatically to optimize for searching with short sequences. A short query is more likely to occur by chance in the database. Therefore increasing the Expect value threshold, and also lowering the word size is often necessary before results can be returned. Low Complexity filtering has also been removed since this filters out larger percentage of a short sequence, resulting in little or no query sequence remaining. Also for short protein sequence searches the Matrix is changed to PAM-30 which is better suited to finding short regions of high similarity.

The databases that can be searched by the BLAST program is user selected, and is subject to frequent updates at NCBI. The most commonly used ones are:

Nr: All non-redundant GenBank CDS translations+PDB+SwissProt+PIR+PRF;

Month: All new or revised GenBank CDS translation+PDB+SwissProt+PIR+PRF released in the last 30 days;

Swissprot: Last major release of the SWISS-PROT protein sequence database (no updates);

Drosophila genome: Drosophila genome proteins provided by Celera and Berkeley Drosophila Genome Project (BDGP);

S. cerevisiae: Yeast (Saccharomyces cerevisiae) genomic CDS translations;

Ecoli: Escherichia coli genomic CDS translations;

Pdb: Sequences derived from the 3-dimensional structure from Brookhaven Protein Data Bank;

Alu: Translations of select Alu repeats from REPBASE, suitable for masking Alu repeats from query sequences. It is available by anonymous FTP from the NCBI website. See “Alu alert” by Claverie and Makalowski, Nature vol. 371, page 752 (1994).

Some of the BLAST databases, like SwissProt, PDB and Kabat are complied outside of NCBI. Other like ecoli, dbEST and month, are subsets of the NCBI databases. Other “virtual Databases” can be created using the “Limit by Entrez Query” option.

The Welcome Trust Sanger Institute offer the Ensembl software system which produces and maintains automatic annotation on eukaryotic genomes. All data and codes can be downloaded without constraints from the Sanger Centre website. The Centre also provides the Ensembl's International Protein Index databases which contain more than 90% of all known human protein sequences and additional prediction of about 10,000 proteins with supporting evidence. All these can be used for database search purposes.

In addition, many commercial databases are also available for search purposes. For example, Celera has sequenced the whole human genome and offers commercial access to its proprietary annotated sequence database (Discovery™ database).

Various software programs can be employed to search these databases. The probability search software Mascot (Matrix Science Ltd.). Mascot utilizes the Mowse search algorithm and scores the hits using a probabilistic measure (Perkins et al., 1999, Electrophoresis 20: 3551-3567, the entire contents are incorporated herein by reference). The Mascot score is a function of the database utilized, and the score can be used to assess the null hypothesis that a particular match occurred by chance. Specifically, a Mascot score of 46 implies that the chance of a random hit is less than 5%. However, the total score consists of the individual peptide scores, and occasionally, a high total score can derive from many poor hits. To exclude this possibility, only “high quality” hits—those with a total score>46 with at least a single peptide match with a score of 30 ranking number 1—are considered.

Other similar software can also be used according to manufacturer's suggestion.

PubMed, available via the NCBI Entrez retrieval system, was developed by the National Center for Biotechnology Information (NCBI) at the National Library of Medicine (NLM), located at the National Institutes of Health (NIH). The PubMed database was developed in conjunction with publishers of biomedical literature as a search tool for accessing literature citations and linking to full-text journal articles at web sites of participating publishers.

Publishers participating in PubMed electronically supply NLM with their citations prior to or at the time of publication. If the publisher has a web site that offers full-text of its journals, PubMed provides links to that site, as well as sites to other biological data, sequence centers, etc. User registration, a subscription fee, or some other type of fee may be required to access the full-text of articles in some journals.

In addition, PubMed provides a Batch Citation Matcher, which allows publishers (or other outside users) to match their citations to PubMed entries, using bibliographic information such as journal, volume, issue, page number, and year. This permits publishers easily to link from references in their published articles directly to entries in PubMed.

PubMed provides access to bibliographic information which includes MEDLINE as well as:

The out-of-scope citations (e.g., articles on plate tectonics or astrophysics) from certain MEDLINE journals, primarily general science and chemistry journals, for which the life sciences articles are indexed for MEDLINE.

Citations that precede the date that a journal was selected for MEDLINE indexing.

Some additional life science journals that submit full text to PubMed Central and receive a qualitative review by NLM.

PubMed also provides access and links to the integrated molecular biology databases included in NCBI's Entrez retrieval system. These databases contain DNA and protein sequences, 3-D protein structure data, population study data sets, and assemblies of complete genomes in an integrated system.

MEDLINE is the NLM's premier bibliographic database covering the fields of medicine, nursing, dentistry, veterinary medicine, the health care system, and the pre-clinical sciences. MEDLINE contains bibliographic citations and author abstracts from more than 4,300 biomedical journals published in the United States and 70 other countries. The file contains over 11 million citations dating back to the mid-1960's. Coverage is worldwide, but most records are from English-language sources or have English abstracts.

PubMed's in-process records provide basic citation information and abstracts before the citations are indexed with NLM's MeSH Terms and added to MEDLINE. New in process records are added to PubMed daily and display with the tag [PubMed—in process]. After MeSH terms, publication types, GenBank accession numbers, and other indexing data are added, the completed MEDLINE citations are added weekly to PubMed.

Citations received electronically from publishers appear in PubMed with the tag [PubMed—as supplied by publisher]. These citations are added to PubMed Tuesday through Saturday. Most of these progress to In Process, and later to MBDLINE status. Not all citations will be indexed for MEDLINE and are tagged, [PubMed—as supplied by publisher].

The Batch Citation Matcher allows users to match their own list of citations to PubMed entries, using bibliographic information such as journal, volume, issue, page number, and year. The Citation Matcher reports the corresponding PMID. This number can then be used to easily to link to PubMed. This service is frequently used by publishers or other database providers who wish to link from bibliographic references on their web sites directly to entries in PubMed.

As used herein, nr database includes all non-redundant GenBank CDS translations+PDB+SwissProt+PIR+PRF according to the BLAST website.

The E-value for an alignment score “S” represents the number of hits with a score equal to or better than “S” that would be “expected” by chance (the background noise) when searching a database of a particular size. In BLAST 2.0, the E-value is used instead of a P-value (probability) to report the significance of a match. The default E-value for blastn, blastp, blastx and tblastn is 10. At this setting, 10 hits with scores equal to or better than the defined alignment score, S, are expected to occur by chance (in a search of the database using a random query with similar length). The E-value can be increased or decreased to alter the stringency of the search. Increase the E-value to 1000 or more when searching with a short query, since it is likely to be found many times by chance in a given database. Other information regarding the BLAST program can be found at the NCBI BLAST website.

IMAC

The principles of IMAC are generally appreciated. It is believed that adsorption is predicated on the formation of a metal coordination complex between a metal ion, immobilized by chelation on the adsorbent matrix, and accessible electron donor amino acids on the surface of the polypeptide to be bound. The metal-ion microenvironment including, but not limited to, the matrix, the spacer arm, if any, the chelating ligand, the metal ion, the properties of the surrounding liquid medium and the dissolved solute species can be manipulated by the skilled artisan to affect the desired fractionation.

Not wishing to be bound by any particular theory as to mechanism, it is further believed that the more important amino acid residues in terms of binding are histidine, tryptophan and probably cysteine. Since one or more of these residues are generally found in polypeptides, one might expect all polypeptides to bind to IMAC columns. However, the residues not only need to be present but also accessible (e.g., oriented on the surface of the polypeptide) for effective binding to occur. Other residues, for example poly-histidine tails added to the amino terminus or carboxyl terminus of polypeptides, can be engineered into the recombinant expression systems by following the protocols described in U.S. Pat. No. 4,569,794.

The nature of the metal and the way it is coordinated on the column can also influence the strength and selectivity of the binding reaction. Matrices of silica gel, agarose and synthetic organic molecules such as polyvinyl-methacrylate co-polymers can be employed. The matrices preferably contain substituents to promote chelation. Substituents such as iminodiacetic acid (IDA) or its tris (carboxymethyl) ethylene diamine (TED) can be used. IDA is preferred. A particularly useful IMAC material is a polyvinyl methacrylate co-polymer substituted with IDA available commercially, e.g., as TOYOPEARL AF-CHELATE 650M (ToyoSod a Co.; Tolyo. The metals are preferably divalent members of the first transition series through to zinc, although Co⁺⁺, Ni⁺⁺, Cd⁺⁺ and Fe-⁺⁺ can be used. An important selection parameter is, of course, the affinity of the polypeptide to be purified for the metal. Of the four coordination positions around these metal ions, at least one is occupied by a water molecule which is readily replaced by a stronger electron donor such as a histidine residue at slightly alkaline pH.

In practice the IMAC column is “charged” with metal by pulsing with a concentrated metal salt solution followed by water or buffer. The column often acquires the color of the metal ion (except for zinc). Often the amount of metal is chosen so that approximately half of the column is charged. This allows for slow leakage of the metal ion into the non-charged area without appearing in the eluate. A pre-wash with intended elution buffers is usually carried out. Sample buffers may contain salt up to 1M or greater to minimize nonspecific ion-exchange effects. Adsorption of polypeptides is maximal at higher pHs. Elution is normally either by lowering of pH to protonate the donor groups on the adsorbed polypeptide, or by the use of stronger complexing agent such as imidazole, or glycine buffers at pH 9. In these latter cases the metal may also be displaced from the column. Linear gradient elution procedures can also be beneficially employed.

As mentioned above, IMAC is particularly useful when used in combination with other polypeptide fractionation techniques. That is to say it is preferred to apply IMAC to material that has been partially fractionated by other protein fractionation procedures. A particularly useful combination chromatographic protocol is disclosed in U.S. Pat. No. 5,252,216 granted Oct. 12, 1993, the contents of which are incorporated herein by reference. It has been found to be useful, for example, to subject a sample of conditioned cell culture medium to partial, purification prior to the application of IMAC. By the term “conditioned cell culture medium” is meant a cell culture medium which has supported cell growth and/or cell maintenance and contains secreted product. A concentrated sample of such medium is subjected to one or more polypeptide purification steps prior to the application of a IMAC step. The sample may be subjected to ion exchange chromatography as a first step. As mentioned above various anionic or cationic substituents may be attached to matrices in order to form anionic or cationic supports for chromatography. Anionic exchange substituents include diethylaminoethyl (DEAE), quaternary aminoethyl (QAE) and quaternary amine (Q) groups. Cationic exchange substituents include carboxymethyl (CM), sulfoethyl (SE), sulfopropyl (SP), phosphate (P) and sulfonate (S). Cellulosic ion exchange resins such as DE23, DE32, DE52, CM-23, CM-32 and CM-52 are available from Whatman Ltd. Maidstone, Kent, U. K. SEPHADEX.RTM.-based and cross-linked ion exchangers are also known. For example, DEAE-, QAE-, CM-, and SP-dextran supports under the tradename SEPHADEX.RTM. and DEAE-, Q-, CM-, and S-agarose supports under the tradename SEPHAROSE.RTM. are all available from Pharmacia AB. Further both DEAE and CM derivatized ethylene glycol-methacrylate copolymer such as TOYOPEARL DEAE-650S and TOYOPEARL CM-650S are available from Toso Haas Co., Philadelphia, Pa. Because elution from ionic supports sometimes involves addition of salt and IMAC may be enhanced under increased salt concentrations. The introduction of a IMAC step following an ionic exchange chromatographic step or other salt mediated purification step may be employed. Additional purification protocols may be added including but not necessarily limited to HIC, further ionic exchange chromatography, size exclusion chromatography, viral inactivation, concentration and freeze drying.

### EXAMPLE 1

Proof of concept for this tethered molecule proteomics approach has been demonstrated using the well-known anti-cancer agent Methotrexate as the chemical “bait”. Methotrexate (MTX) is a folate antimetabolite that has been used intensively for the treatment of highly proliferative diseases such as, rapidly growing tumors, acute leukemia, rheumatoid arthritis, psoriasis, AIDS-associated pneumocystis carinii and other chronic inflammation disorders. Methotrexate has recognized efficacy as an anticancer, anti-inflammatory and immunosuppressive agent. In cancer, the mechanism of action of Methotrexate is due to cytotoxicity originating from the accumulation of its corresponding polyglutamated metabolites in cells. Methotrexate is taken into cells by reduced folate carrier (RFC) protein, where it is polyglutamated by folylpolyglutamate synthetase (FPGS). Upon polyglutamation, Methotrexate binds to dihydrofolate reductase (DHER), interrupting the conversion of dihydrofolate to the activated N5,N10-methylene-tetrahydrofolate. N5,N10-methylene-tetrahydrofolate is the main methylene donor in de novo purine biosynthesis, providing the methyl group for the conversion of dUMP to deoxythymidilate for DNA synthesis and for many trans-methylation processes. The underlying molecular mechanism of action of Methotrexate in inflammation and immunosupression remains unclear, despite its wide use.

The three main targets of antifolate drugs in the clinic are dihydrofolate reductase (DHFR), thymidylate synthase (TS) and glycinamide ribonucleotide transformylase (GART). Several newer-generation classical and non-classical antifolate drugs (non-polyglutames) are now under evaluation in the clinic and show promising results. It has been established that Methotrexate and other antifolates bind other proteins, for example amino-imidazolecarboxamide-ribonucleotide transformylase (AICART), serine hydroxymethyltransferase (SHMT), folylpolyglutamyl synthetase (FPGS), gamma-glutainyl hydrolase (gamma-GH), and folate transporters (RFC).

The main problem with classical antifolates is that accumulation of polyglutamated metabolites causes drug resistance in cells. Several mechanisms of resistance have been identified, including defective transport through cell membranes, amplification of dihydrofolate reductase, reduced expression of FPGS and upregulation of γ-glutamyl hydrolase, all of which have been proposed as the underlying basis for the mechanism of resistance to Methotrexate. Because of this increased resistance there is a need for new drugs that could be used in combinatory therapies with current antifolate drugs. The new drugs in such a “drug cocktail” would not only target the main pathways but also, any salvage pathways responsible for Methotrexate resistance. The development of diagnostic markers for antifolate drug resistant tumors would also be beneficial in deciding which therapies to choose for those tumors. Equally important is an understanding of the underlying molecular mechanism of action and toxicity of existing and emerging antifolate therapeutics.

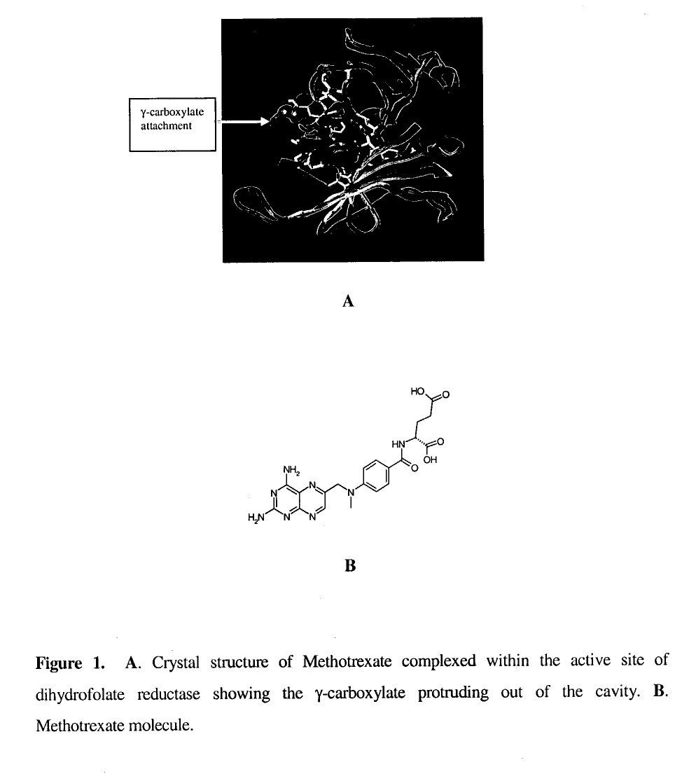

From a structural point of view Methotrexate is one of the most studied drugs in the literature. A search in the protein data bank for the keyword Methotrexate resulted in 62 entries. Most of these entries are for Methotrexate or derivatives in complexes with DHFR or DHFR mutants from different species, but structures for TS also exist. The crystal structure of GART in complex with a molecule of Glycinamideribonucleotide (GAR) and a folate analog is also available. In these structures the aminopterin and the alpha carboxylate groups of the molecule are buried inside the binding site and make key hydrogen bond interactions with the protein, while the gamma carboxylate group protrudes out of the cavity (FIG. 1).

For the proof-of-concept experiment commercially available Methotrexate bound to an agarose support was used. This material is a mixture resulting from linkage to the support through the alpha- and gamma-carboxylates of the molecule. From the structures of Methotrexate complexes only the gamma carboxylate-linked material is capable of binding proteins from a cell lysate, as the linkage through the alpha carboxylate is sterically hindered.

Protocol:

Preparation of cell lysates: HEK 293 cells (typically 10⁷) were harvested, washed with PBS, then lysed in a buffer containing 20 mM Tris, 150 mM NaCl, 1% NP-40, 0.5% sodium deoxycholate supplemented with protease inhibitors. After incubation for 30 minutes at 4° C. with shaking, the lysates were clarified by centrifugation (27,000×g). In some experiments, cells were lysed using 20 strokes of a Dounce® homogenizer in the absence of detergents. Although similar results were obtained, detergent-based lysis was most-often used. In most cases, proteins in the clarified lysate were directly applied to Methotrexate-affinity columns. While optimizing the protocol, however, several experimental variations were tested on cell lysates including concentration by ammonium sulfate precipitation, or removal of nucleic acid with Streptomycin sulfate. In such cases, the protein sample was desalted using a PD 10 protein-desalting column (Pharmacia), which had been pre-equilibrated in the same buffer (10 mM potassium phosphate pH 7.5)

Affinity Chromatography: The desalted lysate was loaded onto a column of pre-equilibrated MTX-agarose (Sigma, 50 μL bed volume) or sepharose 4B agarose as a negative control. The lysates were allowed to slowly flow through the matrix under gravity flow. The columns were then washed with 4×0.6 mls of the same potassium phosphate buffer with various concentrations of NaCl (usually 0.4 M but occasionally 1.0 M), followed by a quick rinse with 0.2 mls of potassium phosphate (0.1 M, pH 6.0)+100 mM NaCl, and eluted with 2×100 μl of 10 mM Methotrexate in potassium phosphate (0.1 M, pH 5.6)+100 mM NaCl. Eluates containing the proteins eluted by Methotrexate were then concentrated by spinning through microcon 3 (from Amicon). Retentates from the microcons were then loaded onto SDS-PAGE 4%-15% gradient mini gels (Bio-Rad). Gels were stained with Gel Code Blue (Pierce), de-stained and imaged. Bands of interest were excised, diced, trypsin digested, and sent for mass spectrometry (MS) analysis.

Protein Identification by Mass Spectrometry: Tryptic peptides were recovered from individual gel bands or using the gel free method disclosed in co-pending application U.S. Ser. No. 60/343,859 (filed Dec. 28, 2001, entire content incorporated by reference herein). The peptides were then separated by reverse phase chromatography on C18 resin and directly injected into a mass spectrometer using an automated sample-loading device from 96 well plates. Two types of mass spectrometry platforms were used: 1) quadrupole ion traps (LCQ Deca, Thermo Finnigan), and 2) customized quadrupole time-of-flight (TOF) hybrid instruments (QSTAR Pulsar, MDS Sciex). Both were operated in data-dependent mode, which produces tandem MS spectra (MS/MS) of all peptide species present above a programmed threshold. The spectra generated were analyzed on a custom-built multi-node server platform (RADARS, ProteoMetrics), which uses two database searching programs, Sonar (ProteoMetrics) and Mascot (Matrix Sciences). The identities of the proteins were obtained from database queries of the MS derived data. The databases searched included NCBI non-redundant (nr) protein, EMBL Ensemble predicted protein, NCBI human chromosomal, and proprietary internal databases.

Docking studies: Protein X-ray crystal structure coordinates were downloaded from public (or proprietary private) protein data banks. The corresponding pdb codes (www.rcsb.org/pdb) for the proteins used for the docking study are given in Table 2. All waters of crystallization were removed and all protein hydrogens were added. Kollman charges were used for all protein atoms using SYBYL (Tripos, St. Louis, Mo.) and the protein file saved as a sybyl mol2 file. The initial conformation of the Methotrexate was extracted from the crystal structure complex of dihydrofolate reductase and Methotrexate (PDB code 1rg7). Coordinates for the molecule were extracted and the atom types checked and corrected and all hydrogens and Gasteiger-Huckel charges were added. Methotrexate was reverse docked into coordinates of all proteins listed in Table 3 using the standard default settings of the program GOLD (CCDC, Cambridge, UK). Binding modes were visually inspected in search of acceptable poses where the gamma carboxylate of Methotrexate protruded out of the binding site as observed for DHFR and could be considered compatible with binding.

Results:

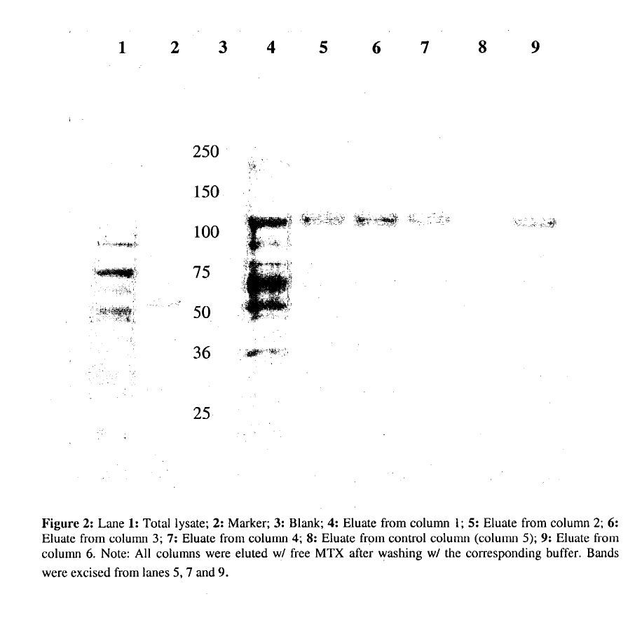

FIG. 2 is a gel image showing the eluates from the six columns. Table 1 shows the wash and elution conditions used for each column.

TABLE 1
Column Wash and Elution conditions

| | | | Rinse | Elution |
|---|---|---|---|---|
| Column | | Wash Buffer | Buffer | Buffer |
| # | Matrix | (NaCl conc, pH) | (pH) | (pH) |
| 1 | MTX-Agarose | 100 mM, pH 7.5 | 6.0 | 5.6 |
| 2 | MTX-Agarose | 200 mM, pH 7.5 | 6.0 | 5.6 |
| 3 | MTX-Agarose | 300 mM, pH 7.5 | 6.0 | 5.6 |
| 4 | MTX-Agarose | 400 mM, pH 7.5 | 6.0 | 5.6 |
| 5 | Sepharose 4B | 400 mM, pH 7.5 | 6.0 | 5.6 |
| 6 | MTX-Agarose | 400 mM, pH 7.5 | 7.5 | 7.5 |

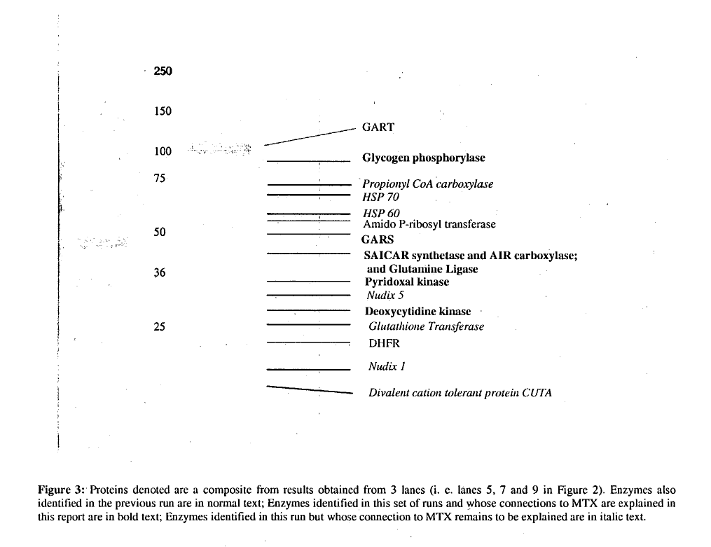

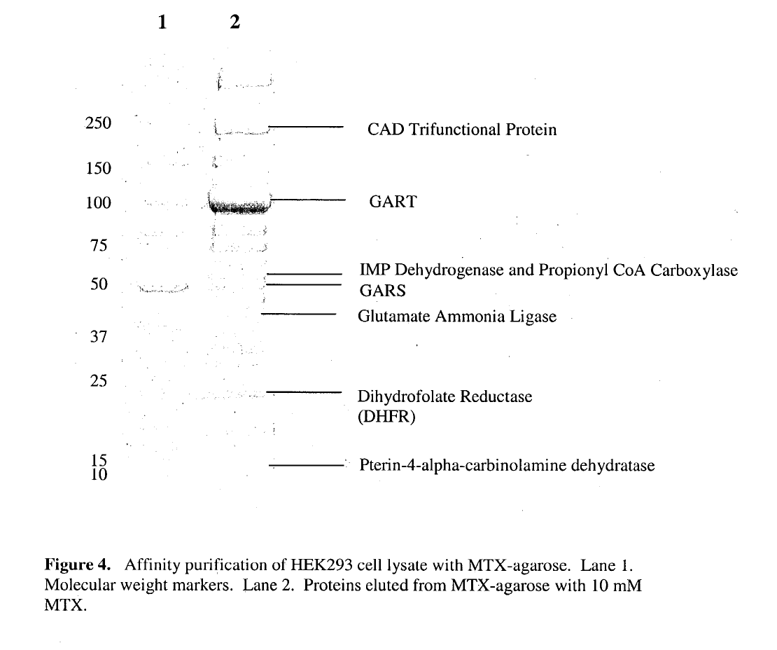

FIGS. 3 and 4 show proteins identified by mass spectroscopy denoted on the gel image. The lane seen corresponds to lane 7 from the previous gel image. Table 2 lists the proteins identified by MS.

The information obtained by these experiments has relevance to the design of next-generation folate drug analogues, of which there are several in the clinic. Most folate analogs in the clinic are very cytotoxic. Knowing all the targets of these inhibitors is key to designing less toxic drugs.

TABLE 2
Proteins identified by Mass Spec.

| | Known | | | |
|---|---|---|---|---|
| | Folate | New | | PDB |
| Protein Identified | targets | interactor | MTX | codes |
| Dihydrofolate reductase (DHFR) | ✓ | | | 1RG7 |
| Thymidine Synthetase (TS) | ✓ | | | 1AXW |
| Glycinamideribonucleotide | ✓ | | | 1CDE |
| transformylase (GART) | | | | |
| aminoimidazole ribonucleotide | | | | 1CLI |
| synthetase (AIRS) | | | | |
| Glycinamideribonucleotide | | | | 1GSO |
| synthase (GARS) | | | | |
| Amido | | ✓ | | 1AO0 |
| phosphoribosyltransferase | | | | |
| AIR carboxylase | | | | 1D7A |
| SAICAR synthetase | | | | 1A48 |
| Hypoxanthine | | ✓ | | 1D6N |
| phosphoribosyltransferase | | | | |
| (HPRT) | | | | |
| Deoxycytidine Kinase | | | | Unknown |
| Deoxyguanosine kinase | | ✓ | | 1JAG |
| Pyridoxal Kinase | | ✓ | | 1LHR |
| Glutamate-Ammonia Ligase | | | | 1F52 |
| (Glutamine synthase) | | | | |
| Inosine monophosphate | | ✓ | | 1LON |
| dehydrogenase | | | | |
| Pterin-4-alpha-carbinolamine | | ✓ | | 1DCP |
| dehydrogenase (PCD) | | | | |
| Nudix 1 | | | | Unknown |
| Nudix 5 | | | | 1KHZ |
| Divalent Cation tolerant protein | | | | 1KR4 |
| CUTA | | | | |
| Glutathione synthase | | | | 1GSA |
| Glycogen Phosphorylase | | ✓ | | 1GGN |
| Propionyl CoA carboxylase | | | | Unknown |

Proteins recovered from the Methotrexate matrix were resolved by SDS-PAGE, visualized by staining and identified by mass spectrometry analysis. Proteins will associate with the immobilized ligand either by direct binding, or by interaction with a directly-binding protein. As expected, DHFR was identified as a Methotrexate-associated protein. The presence of a band corresponding to DHFR is confirmation that the column format was adequate and capable of isolating other Methotrexate binding proteins. Further, as an inherent feature of mass spectrometry analysis, strong interactions or over abundant interacting proteins will consistently pass the rigors of the stringent protein identification quality control process. As such, DHFR was used as an internal control (see FIGS. 3 and 4) for which optimized recovery conditions were established.

Interestingly, an enzyme involved in the production of a consumable molecule used in nucleotide synthesis, glutamate ammonia ligase (which supplies glutamine for the de novo purine synthesis) was also found. Deoxycytidine kinase and deoxyguanosine kinase are also involved in DNA synthesis. Other proteins consistently found were Pterin-4-alpha-carbinolamine dehydrogenase (PCD), nudix 1 and nudix 5, CUTA, pyridoxal kinase, glycogen phosphorylase and glutathione synthase.

Discussion:

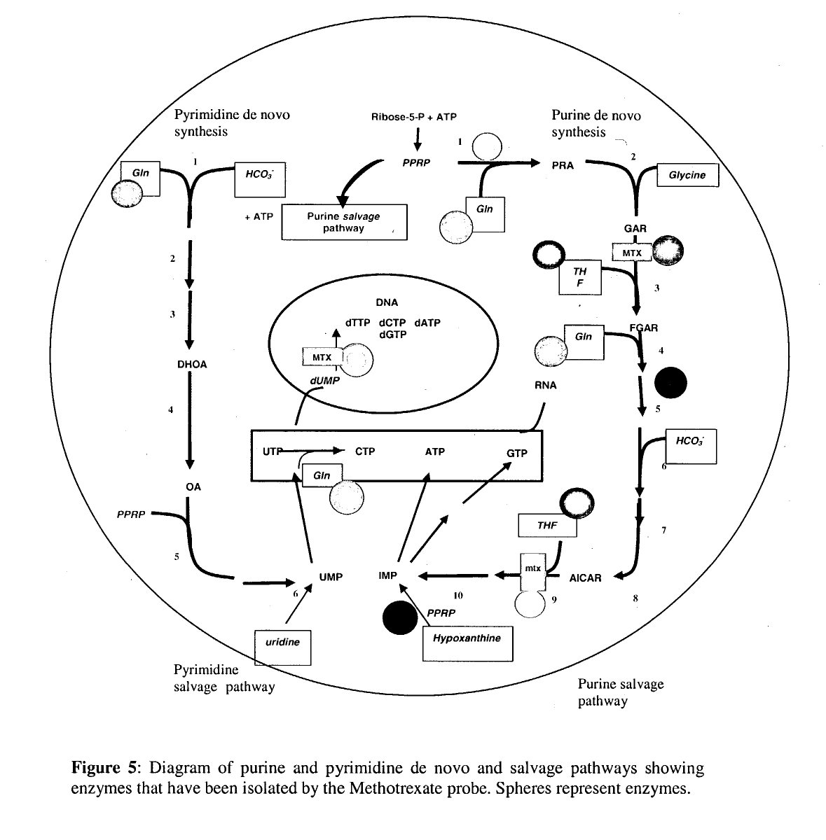

Some of the enzymes identified belong to the same purine biosynthesis pathway as GART and Amido phosphoribosyltransferase. The purine biosynthesis pathway is shown in FIG. 5. As can be seen from this Figure, the validity of hits like GARS, Phosphoribosyl aminoimidazole carboxylase (AIR carboxylase) and Phosphoribosyl aminoimidazole succinocarboxamide synthetase is self-evident. Glutamine ammonia ligase is another enzyme associated with this complex, given the requirement for glutamine by both amido phosphoribosyl transferase as well as phosphoribosyl formyl glycinamide synthase in this de novo purine synthesis pathway.

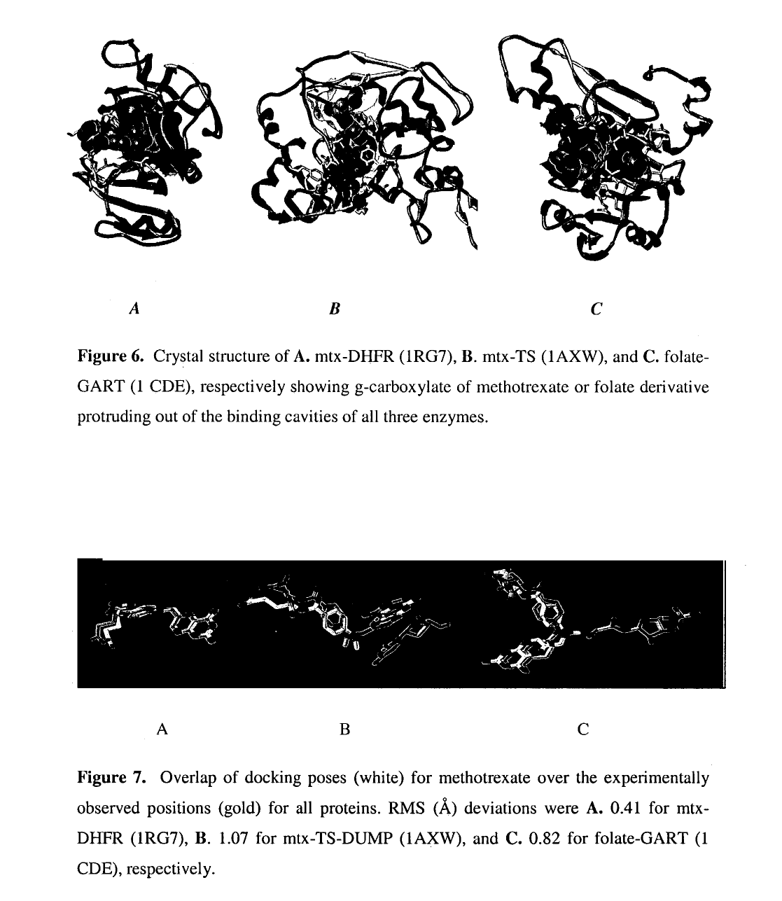

The binding of deoxycytidine kinase, an enzyme that is crucial for sensitivity of cells towards anticancer nucleoside analogues, can also be explained. Deoxycytidine kinase catalyzes the step converting 2′-deoxycytidine to 2′-deoxycytidine-5-phosphate, this in turn is converted into 2′-deoxy-5-hydroxymethyl cytidine-5′-phosphate by the enzyme deoxycytidylate hydroxy methyltransferase (see FIG. 6). This second enzyme is a folate-requiring enzyme, which suggests that the isolation of deoxycytidine kinase is the result of an indirect interaction with Methotrexate.

Another consistent hit observed is Pyridoxal kinase, which catalyzes the conversion of pyridoxal to pyridoxal-5′-phosphate (PLP). PLP is a very important cofactor used by a variety of enzymes involved with diverse reactions such as decarboxylations, deaminations, transaminations, racemizations and aldol cleavages (Stryer L (1988), Biochemistry 3^{rd} Ed., W. H. Freeman and Co. New York). The presence of pyridoxal kinase in these pull down experiments may be explained through the role of PLP in the reaction catalyzed by the enzyme serine hydroxymethyltransferase (SHMT). PLP is a cofactor for SHMT which acts at the step downstream of DHFR, converting the tetrahydrofolate (THY) produced by DHFR into methylene THF, which reaction results in the conversion of Serine to glycine. Pyridoxal kinase could therefore conceivably be in a complex with SHMT. Alternatively, the observed levels of intensity of pyridoxal kinase in all the five MTX-agarose lanes (FIG. 2) suggest a more direct interaction. Relative to pyridoxal kinase, none of the other bands of comparable intensity (or better) in any of the lanes in that gel, proved to be SHMT. This would be the expectation if SUMT were the enzyme that was directly interacting with Methotrexate. The isolation of pyridoxal kinase also explains the identification of glycogen phosphorylase, which is another PLP requiring enzyme.

Another protein identified in the pull down in lane 9 was hypoxanthine phosphoribosyl transferase (HPRT). This enzyme is part of the purine salvage pathway and is responsible for catalyzing the formation of inosinate from PRPP and hypoxanthine. PRPP is the substrate for amido phosphoribosyl transferase which is the first dedicated step in the de novo purine synthesis pathway seen in FIG. 5. Deficiency in HPRT is known to result in higher levels of PRPP and an “acceleration of purine biosynthesis by the de novo pathway” (Stryer L (1988), ibid, 6-499 and 620-621)). In addition, the effect of Methotrexate on raising the intracellular levels of PRPP has been documented (Fung et al., (1996), Oncology 53 (1): 27-30). This same study also demonstrated that hypoxanthine reversed the effect of Methotrexate.

Known Targets of Methotrexate

The nucleotide de novo and salvage pathway proteins were identified in these experiments. Remarkably, a great number of enzymes involved in these pathways, as well as several enzymes not directly dependent on folate cofactors, were identified. This indicates this metabolic pathway is effectively scaffold together through protein-protein interactions, possibly as a means to facilitate forms of co-regulation of the constituent enzymes and achieve a more efficient anabolic process, as described below. This is consistent with paradigms in both signal transduction pathways, and pathways for macromolecular biosynthesis, such as DNA replication and transcription.

As expected, dihydrofolate reductase (DHFR) was identified as a strongly staining band in the gel. This indicated that the column format and protocol were compatible with efficient binding of proteins to the supported Methotrexate molecule. Addition of deoxyuridine 5′-monophosphate (dUMP) to the medium facilitated the recovery of another Methotrexate target, Thymidine Synthetase (TS). TS catalyses the reductive methylation of dUMP to deoxythymidine-5′-monophosphate (dTMP), which is later phosphorylated to dTTP for incorporation into DNA. This is a key step in DNA synthesis and the only pathway to dTMP. This protein is a major target of several anticancer agents such as the widely used dUMP derivative anticancer agent 5-flourouracil (FU). The association of Glycinamideribonucleotide transformylase (GART) with the Methotrexate matrix was not surprising, as it is one of two folate-dependent enzymes in the de novo purine synthesis. Hence, it appears that this association is the consequence of a direct interaction between GART and the Methotrexate ligand. This enzyme catalyses the transfer of a formyl group from 10-formyltetrahydrofolate to the amino group of glycinamide ribonucleotide (GAR). Over the last decade or so, GART has become and important target for anticancer therapy. All three of these proteins are widely studied, and crystal structures with Methotrexate or folate analogs were available; inspection of these structures indicated that Methotrexate could easily bind to these proteins.

Protein-Methotrexate Docking

The Methotrexate-associated proteins identified in this experiment can be separated into two categories (as described above), namely direct binders of the Methotrexate probe or secondary interactors (that is, proteins which interact with direct binders). Since the crystal structures of many of the recovered Methotrexate-associated proteins are available in the pdb, we decided that a good strategy for categorizing the proteins into direct or indirect binders would be to perform in silico protein-ligand docking experiments to investigate the possibility of binding in the proper orientation and compatible with the modified Methotrexate ligand employed in the affinity chromatography procedure, as explained below.

Crystal structures of DHFR, TS and GART (FIG. 7) exist as complexes with Methotrexate or folates, and these were used to validate this approach. Inverse docking of Methotrexate into the binding site of all three proteins was performed and the best 10 docking poses for each investigated.

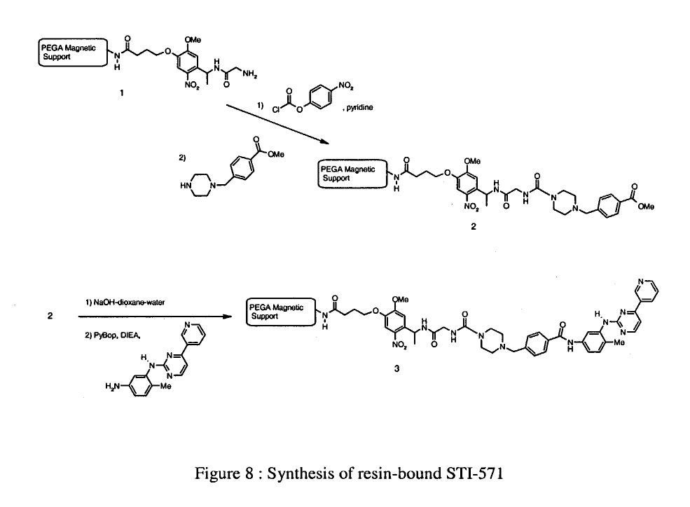

In all cases several poses were found which reproduced the experimentally observed ones. The pose with the greatest overlap over the experimentally observed position was taken as correct and the root mean square (RMS) deviation from the experimentally observed positions was measured. RMS (Å) deviations were: 0.41 for Methotrexate-DHFR (1RG7), 1.07 for Methotrexate-TS (1AXW), and 0.82 for folate-GART (1CDE), respectively. FIG. 8 shows the overlap between the acceptable poses and the experimental positions for all three proteins. In all three cases the docking runs reproduce binding conformations with high fidelity, validating the power of the docking procedure.

Based upon these results it is to be expected that docking runs on other proteins would also generate reasonable solutions. This validation exercise indicated that docking is indeed a useful tool in rationalizing the type of binding interactions responsible for the recovery of the Methotrexate-associated proteins. Whenever a crystal structure was available from the pdb for the proteins identified in our experiments, visual inspection of the structure followed by protein ligand docking with Methotrexate was performed.

New Targets of Methotrexate

Several new interactors were found which directly interacted with the Methotrexate probes. For most of these there is circumstantial evidence in the literature for binding by folates, by Methotrexate or Methotrexate-derivatives, or by chemotypes that can make similar hydrogen bonding interactions as the aminopterin group of Methotrexate. Structural analysis, where the crystal structure was available, followed by docking experiments corroborated this hypothesis for the cases presented next.

Amido phosphoribosyltransferase: This target was found to interact with Methotrexate, even though it is a low abundant protein; it was found in experiments carried out using lysates from four different cell lines, namely BEK293, Jurkat, K562 and A431. Amido phosphoribosyltransferase catalyses the committed step in purine biosynthesis. This enzyme catalysis the addition of an amine group to phosphoribosylpyrohosphate (PPRP). This enzyme is subject to feedback inhibition by end products of the pathway AMP, GMP and IMP through interaction at an allosteric binding site. There is evidence in the literature that Methotrexate inhibition of purine de novo synthesis in leukemia cells occurs before the folate dependent steps carried out by GART and AICART. On treatment with Methotrexate the de novo pathway is completely blocked, accumulation of GAR and AIRCAR intermediates are minimal, whilst accumulation of 5-phosphoribosyl-1-pyrophosphate is 3-4 fold. This is consistent with the interpretation that amido-phosphoribosyltransferase that is being inhibited. Further, in vitro assays performed with MTX-Glu5, the active metabolite of Methotrexate, in cells showed that amido-phosphoribosyltransferase is inhibited. A more recent study, in mitogen stimulated T-lymphocytes, concluded that it is this step which is blocked by Methotrexate. The authors postulate that this could be the underlying mechanism for the efficacy of Methotrexate in Rheumatoid Arthritis. The fact that this enzyme was consistently isolated by its direct interaction with Methotrexate, under a variety of conditions, provides strong evidence of its direct inhibition by Methotrexate. Docking experiments with amido phosphoribosyl-transferase further corroborate this conclusion. Docking Methotrexate in the allosteric GMP binding site of amido phosphoribosyltransferase (PDB code 1AO) resulted in several binding modes that are consistent with binding. The finding that the inhibition of amidophosphoribosyltransferase by Methotrexate is indeed responsible for the efficacy of this drug in Rheumatoid Arthritis is of note, introducing the possibility of new drug chemotypes that are less prone to resistance.

Inosine monophosphate dehydrogenase (IMPDH): IMPDH catalyses the nicotinamide adenosine dinucleotide dependent conversion of Inosine 5′-phosphate to xanthosine 5′phosphase, the first step in the de novo synthesis of guanine nucleotides. Rapid proliferating cells such as lymphocytes depend on the availability of nucleotide pools. It is known that the activity of IMPDH is higher in rapid proliferating cells. Because of these cell requirements, IMPDH is being pursued as a target for immunosuppressive, anticancer and antiviral therapies and several IMPDH inhibitors are now being evaluated in the clinic. Since this enzyme binds the inosine moiety, and other enzymes that bind IMP have been known to also bind folate analogues, it appears that Methotrexate binds this enzyme directly. Docking poses generated also support this conclusion, as several modes that would not interfere with binding were found. The efficacy of Methotrexate as an immunosuppressive agent may be caused at least in part through the direct inhibition of IMPDH.

Hypoxanthine-guanine phosphoribosyltransferase (HPRT): Hypoxanthine-guanine phosphoribosyltransferase is the most important enzyme of the salvage pathway. This. enzyme catalyses the salvage conversion of hypoxanthine and guanine to IMP to GMP respectively, by facilitating the addition of the bases to the activated PPRP molecule. This enzyme, like amido-phosphoribosyltransferase, is involved in amine addition to the PPRP. The activity of salvage enzymes like HPRT is higher than the activity of enzymes involved in the de novo pathways. Agents such as Methotrexate, believed to act primarily on de novo enzymes, are effective in spite of the presence of highly active salvage enzymes. This has recently been accounted for, at least in part, by new observations showing that Methotrexate can reduce the activity of HPRT. Other observations corroborate the in vivo inhibition of HPRT; for example, deficiency in HPRT is known to result in higher levels of PRPP and an acceleration of purine biosynthesis by the de novo pathway. Treatment with Methotrexate also produces an increase on levels of PRPP and this effect is reversible upon treatment with hypoxanthine. These results and our findings, point to direct in vivo inhibition of HPRT by Methotrexate. Our docking experiments are also consistent with direct binding as Methotrexate can fit in the binding pocket of HPRT (1D6N) with good overlap over the positions occupied by hypoxanthine monophosphate with the glutamate group of Methotrexate protruding out of the cavity. Direct inhibition of HPRT could contribute in part the efficacy of Methotrexate as an anti-cancer agent.

Pterin-4-alpha-carbinolamine dehydratase (PCD): Pterin-4-alpha-carbinolamine dehydratase (PCD) catalyses the dehydration of 4a-hydrozytetrahydrobiopterins to the corresponding dihydropterins. Dihydrobiopterin is a substrate of pteridine reductase, an enzyme known to bind Methotrexate directly. The experiments described herein show that Pterin-4-alpha-carbinolamine dehydratase binds directly to Methotrexate. Docking experiments on the structure of Pterin-4-alpha-carbinolamine dehydratase from the crystallographic complex with biopterin (1DCP) supports this conclusion, since several docking poses were found where the pterin moiety of Methotrexate exactly overlaps the biopterin molecule in the complex.

Glycogen phosphorylase: This enzyme is involved in glycogen metabolism, which regulates blood glucose levels and is an important therapeutic target for diabetes. It catalyses the phosphorylitic cleavage of glycogen to glycogen-phosphate. This enzymatic reaction uses pyridoxal phosphate (PLP), a derivative of vitamin 6. Methotrexate, 3′-chloro- and 3′,5′-dichloroMethotrexates and various folate derivatives have been shown to be reversible inhibitors of muscle glycogen phosphorylase b. The experiments described herein show that glycogen phosphorylase is a direct binder of Methotrexate. Docking experiments on the structure of glycogen phosphorylase (1GGN) also corroborates this hypothesis, as Methotrexate in several of the docking poses is found with the g-carboxylate protruding out of the cavity.

Pyridoxal kinase: This enzyme catalyzes the conversion of pyridoxal to pyridoxal-5′-phosphate (PLP). PLP is an important cofactor in a variety of reactions such as decarboxylations, deaminations, transaminations, racemizations and aldol cleavages. The experiments described herein show that Pyridoxal kinase is a direct binder of Methotrexate. The crystal structure of pyridoxal kinase was recently solved, but the coordinates are not yet available. Alkylxanthines are competitive inhibitors of Pyridoxal kinase; as already argued earlier (see section on HPRT), the pterin group of Methotrexate can act as a substitute of the xanthine moiety. Furthermore, extensive medicinal chemistry work done on antimetabolite research has elucidated that the pterin ring can be replaced with xanthine and xanthine-like moieties. Examples of this are Pemetrexed, (ALIMTA, LY-231514) the classical antimetabolite TS inhibitor drug from Lilly and Tomudex (ZD9331) the non-classical TS inhibitor from AstraZeneca. The fact that another PLP dependent enzyme, glycogen phosphorylase, binds Methotrexate further corroborates that pyridoxal kinase is binding through a direct interaction with the tethered Methotrexate molecule.

Deoxycytidine kinase and deoxyguanosine kinase: These enzymes are members of the deoxyribonucleoside kinases that phosphorylate deoxyribonucleosides, a crucial reaction in the biosynthesis of DNA precursors through the salvage pathway. These kinases are of therapeutic interest as they are crucial in the activation of a number of anticancer and antiviral drugs, such as 2-chloro-2′-deoxyadenosine, azidothymidine and acyclovir. The crystal structure of deoxycytidine kinase is not known, but that of deoxyguanosine kinase is (1JAG), and was used in docking experiments. Docking into the active site of deoxyguanosine kinase produced binding modes consistent with direct binding. Most poses placed the Methotrexate molecule in a configuration that extended the γ-carboxylate out of the cavity. The experiments described herein show that this kinase binds to Methotrexate through a direct interaction.

Aminoimidazoleribonucleotide carboxylase: Air carboxylase catalyses the carboxylation of aminoimidazoleribonucleotide. The domain associated with this enzymatic activity in animals is part of a bifunctional polypeptide containing SAICAR synthase and air carboxylase. In the experiments described herein a single band contained peptides from both domains of the bifunctional enzyme. The crystal structure of Air carboxylase (1D7A) is available from the protein databank in complex with amidoimidazole-ribonucleotide (Air). Docking runs of Methotrexate in the air binding-site resulted in several poses compatible with binding. In these poses the pterin moiety of Methotrexate is perpendicular to the imidazole ring of Air, but the gamma carboxylate does protrude out of the cavity. These experiments support the conclusion that this protein was associated indirectly with Methotrexate, as the result of direct inhibition of GART.

Phosphoribosylaminoimidazolesuccinocarboxamide (SAICAR) synthase: this enzyme catalyses the seventh step in the biosynthesis of purine nucleotides. The crystal structure of SAICAR synthase reveals that the active site is a very open cleft. There is no precedence for direct binding of SAICAR to folates or Methotrexate. Docking experiments resulted only in poses in which the complete Methotrexate molecule is buried deep into the cleft. In all poses both carboxylate groups are involved in hydrogen bonding interactions and fully buried inside the protein and would therefore interfere with binding to the attached Methotrexate.

GARS: In humans, the second, third and fifth steps of de novo purine biosynthesis are catalyzed by a trifunctional protein with glycinamide ribonucleotide synthetase (GARS), aminoimidazole ribonucleotide synthetase (AIRS) and glycinamide ribonucleotide formyltransferase (GART) enzymatic activities. GARS catalyzes the second step of the de novo purine biosynthetic pathway, the conversion of phosphoribosylamine, glycine, and ATP to glycinamide ribonucleotide (CAR), ADP, and Pi. In the experiments described herein GARS-derived peptides were isolated both as part of the trifunctional protein GARS-AIRS-GART (at its predicted Mᵣ of 110 kDa), and also as a separate band of Mᵣ 50 kDa in the gel. Transfection of Chinese hamster ovaries (CHO) cells with the human GARS-AIRS-GART gene has shown that this gene encodes not only the trifunctional protein of 110 kDa but also a monofunctional GARS protein of 50 kDa produced by alternative splicing, resulting in the use of a polyadenylation site in the intron between the terminal GARS and the first AIRS exons. The mechanism of Methotrexate binding was also, investigated by docking experiments on the crystal structure of GARS. This protein, like SAICAR synthase has a very large open binding site, and no docking conformations were found where Methotrexate could form productive stable complex with GARS. Although GART and GARS are part of the same trifunctional protein, there may be a protein-protein docking interaction between the domains. Protein-protein interactions between the first and second enzymes in purine biosynthesis, Amidophosphoribosyltransferase and GARS, have also been postulated. Phosphoribosylamine is the product of the first enzyme and the substrate for the next reaction in the purine biosynthesis chain of events. There is evidence that this phosphoribosylamine reagent transfer occurs from one enzyme to the next via a coupling between Amidophosphoribosyltransferase and GARS, rather than through free diffusion. This presents a second possible mechanism for the association of GARS with Methotrexate.

Phosphoribosylaminoimidazole synthetase (AIRS): This enzyme is part of the trifunctional, GARS-AIRS-GART protein. Peptides for all three domains were found in the same band in the gel. Docking runs on the crystal structure of AIRS (1CLI) does not indicate direct binding with the Methotrexate probe. We postulate that the presence of this enzyme is simple due to the fact that it is part of the trifunctional protein GARS-AIRS-GART and that binding occurs through the GART domain.

Gluthathione synthase: Interestingly, glutathione synthase is structurally related to SAICAR synthase. Structural comparisons of these two proteins reveal a common fold. This fold is also shared with heat shock protein HSP70. The crystal structure of glutathione synthase is available (1GSA) and was used in Docking exercises that were inconclusive. In all docking modes the complete Methotrexate molecule is buried deep within a very closed active site. Structural rearrangement of the protein would open the site, as required for the substrate to bind to the protein. Such opening of the site could produce a conformation consistent with direct binding; however without an available crystal structure this is difficult to confirm.

Nudix 1 and 5: Nudix hydrolases are housekeeping proteins involved in the hydrolysis of nucleoside phosphates. Nudix-1 (MTH1), for example, hydrolyses 8-oxo-dGTP and thus avoids errors caused by their misincorporation during DNA replication or transcription, which can result in carcinogenesis or neurodegeneration. Nudix 5 hydrolyses ADP sugars to AMP and sugar-5-phosphates. Nudix hydrolases that degrade dinucleoside and diphosphoinositol polyphosphates also have 5-phosphoribosyl 1-pyrophosphate (PRPP) pyrophosphatase activity that generates the glycolytic activator ribose 1,5-bisphosphate. The fact that these enzymes bind nucleotides and PRPP, two substrates already encountered in several other of the targets believed to be direct interactors of Methotrexate, and their role in purine and pyrimidine synthesis, is significant. Several crystal structure examples of ADP nudix hydrolases are available in the protein databank, but none that represent 8-oxo-dGTP hydrolase. We obtained the crystal structure of an ADP nudix hydrolases (nudix 5, 1 KHZ) and docked Methotrexate into the nucleotide binding site. Interestingly, poses of Methotrexate were found that are consistent with a direct interaction. The glutamate group can protrude out of the cavity, while the aminopterin group is buried well within the binding site, making strong hydrogen bonding interactions. Although there is no evidence in the literature that nudix hydrolases bind folates or Methotrexate, we believe that the presence of these proteins (at least nudix 5) in our gels results from direct interactions with the Methotrexate probe.

Finally, propionyl CoA carboxylose and divalent cation tolerant protein CUTA are enzymes that are pulled down consistently. A literature search does not show previous evidence of any interaction between Methotrexate and these enzymes.

Conclusion:

Methotrexate is an important drug with applications in several therapeutic areas with unmet medical needs. The efficacy of this drug in many cases has been arrived at serendipitously. Although, it has been widely used in rheumatoid arthritis (RA) and immunosuppression; a clear mechanism of action is not yet available. We were able to identify the three main therapeutic targets of antifolate therapies in the clinic in a single experiment. We show that Methotrexate is able to interact with at least six other proteins not widely regarded as targets of this drug, but with crucial roles in medicine and drug discovery. Inhibition of for IMPDH by Methotrexate, for example, may be the underlying reason behind its efficacy as an immunosuppressive agent. Further, inhibition of the first enzyme in the de novo synthesis of nucleotides, amidophosphoribosyltransferase, may be responsible at least in part for its efficacy in Rheumatoid arthritis.

Another aspect we believe has paramount importance is the capture, in a single experiment, of such a large portion of the de novo and salvage nucleotide synthesis pathways. Seven of the ten steps in purine synthesis are carried out by enzymes identified with our drug probe. This remarkable finding indicates that these proteins, like signal tranduction proteins, are structurally engineered in such a way as to facilitate the transfer of the evolving reagent (purine) from one enzyme to the next via tandem protein protein recognition events. This has been observed already for the channelling transfer of the aminephosphoribosyl molecule from amidophosphoribosyltransferase to glycinamideribosyl synthase for the next reaction in the sequence to take place. Furthermore, the fact that so many of the proteins identified in these experiment represent viable drug discovery targets in the pharmaceutical industry is significant.

This study demonstrates our ability to identify significant portions of pathways which can be affected by a drug or drug candidate. Besides verifying interactions with the intended target, it also succeeded in demonstrating the utility of the approach to discover a host of unknown or undesired interactions. This was proved by the identification of Pyridoxal kinase, an important enzyme whose disruption could result in extensive unintended effects. The fact that a good portion of the hits show that there are indeed interactions between a relatively old anti-cancer agent like Methotrexate and proteins with which there have never been any documented connections, is surprising. Information of this nature could in turn go a long way in helping to explain the side effects of drugs as well as help with evaluating potential drugs for their specificity.

These results demonstrate that our proprietary proteomics technology has an important role to play in the drug discovery process. The findings that such interaction data could be obtained from a single experiment is both surprising and an elegant proof of concept for the invention disclosed herein. It allows an un-biased monitoring of the interactions between a drug and the protein content of a cell. This information is crucial in deepening the understanding of the pharmacology of a drug and aids, form example, in the development of in vitro assays, functional cell assays and markers. This technology has particular promise as a tool to stratify patient populations for clinical studies by developing drug protein fingerprints that can be correlated with patient compliance. Drug response is a very complex event; the proteomics fingerprint of a drug represents a Pharmaco-dynamic/Pharmaco-kinetic filter that allows only relevant proteins to be monitored. By monitoring a full compliment of proteins that interact with a drug the underlying reason for response is better revealed.

### EXAMPLE 2

A second series of experiments were performed using Methotrexate attached to a magnetic support consisting of a polyethylene glycol dimethylacrylamide (PEGA) copolymer (obtained from Polymer Laboratories Limited, Church Stretton, U.K.). Although this polymeric material itself has been successfully used as a matrix for solid phase synthesis and affinity chromatography, a magnetic version based on this material has never been reported. The magnetic version is composed of submicron sized magnetite particles encased in a 150-300 micron sized bead made up of a copolymer of bisacrylamido polyethylene glycol, N,N-dimethyl acrylamide and monoacrylamido polyethylene glycol (PEGA) having an initial loading capacity of 0.1-0.2 mmoles free amine/gram of support. As shown in FIG. 8, the resin bound glycine 1 was then coupled to L-Methotrexate following the standard peptide coupling conditions of Benzotriazole-1-yl-oxy-tris-pyrolidinophosphonium hexafluorophosphate (PyBop) and diisoopropylethylamine (DIEA) in dimethylformamide (DMF) to give the resulting L-methotrexate coupled support 4 as a mixture of alpha and gamma coupled products.

Procedure:

Treatment with lysate from HEK 293 was carried out as in Example 1.

Results and Conclusion:

DHFR, GART and GARS were identified in this experiment, demonstrating the feasibilty of using a small molecule (e.g. a drug or drug candidate) immobilized on a magentic support for the capture of proteins which interact with it.

This novel use of a magnetic support extends the usefulness of the method disclosed herein.

References

Aghi M, Kramm C M, Breakefield X O., J Natl Cancer Inst Jul. 21, 1999;91(14):1233-41.

Aherne G W, Hardcastle A, Ward E, Dobinson D, Crompton T, Valenti M, Brunton L, Jackman A L. Clin Cancer Res September 2001;7(9):2923-30.

Allegra C J, Drake J C, Jolivet J, Chabner B A, Proc Natl Acad Sci U S A August 1985;82(15):4881-5.

Allison A C. Immunopharmacology May 2000;47(2-3):63-83.

Almassy R J, Janson C A, Kan C C, Hostomska Z. Proc Natl Acad Sci USA Jul. 1, 1992;89.

Arlington S A: Industrialization of R&D in the 21st century. ECPI-Barcelona 2001, PricewatersCoopers.

Balendiran G K, Molina J A, Xu Y, Torres-Martinez J, Stevens R, Focia P J, Eakin A E, Sacchettini J C, Craig S P 3rd. Protein Sci May 1999;8(5):1023-31.

Bera A K, Chen S, Smith J L, Zalkin H. J Bacteriol July 2000; 182(13):3734-9.

Brodsky G, Barnes T, Bleskan J, Becker L, Cox M, Patterson D. Hum Mol Genet November 1997;6(12):2043-50.

Chen, S., Tomchick, D. R., Wolle, D., Hu, P., Smith, J. L., Switzer, R. L., Zalkin, H. Biochemistry Sep. 2, 1997;36(35):10718-26.

Chen Z D, Dixon J E, Zalkin H., Proc Natl Acad Sci U S A April 1990;87(8):3097-101.

CHI, Pharmacogenomics/Pharmacoproteomics, Europe. May 2002, Munich, Germany.

Cole P D, Kamen B A, Gorlick R. Banerjee D, Smith A K, Magill E, Bertino J R., Cancer Res Jun. 1, 2001;61(11):4599-604.

Costi M P, Ferrari S., Curr Drug Targets June 2001;2(2):135-66.

Cronk J D, Endrizzi J A, Alber T. Protein Sci October 1996;5(10):1963-72.

Cronstein B N, Rheum Dis Clin North Am November 1997;23(4):739-55.

Fairbanks L D, Ruckemann I C, Qiu Y, Hawrylowicz C M, Richards D F, Swaminathan R, Kirschbaum B, Simmonds H A., Biochem J Aug. 15, 1999;342 (Pt 1): 143-52.

Figeys, D, L D McBroom & MF Moran (2001) Mass spectrometry for the study of protein-protein interactions. Methods (Methods in Enzymology) 24(3): 230-239.

Fisher D L, Safrany S T, McLennan A G, Cartwright J L. J Biol Chem Oct. 4, 2002; [ahead of print].

Fritz T A, Tondi D, Finer-Moore J S, Costi M P, Stroud R M., Chem Biol October 2001;8(10):981-95.

Fung K P, Lam W P, Choy Y M, Lee C Y., Oncology January-February 1996;53(1):27-30.

Gabelli S B, Bianchet M A, Bessman M J, Amzel L M. Nat Strict Biol May 2001;8(5):467-72.

Gabelli, S. B., Bianchet, M. A., Ohnishi, Y., Ichikawa, Y., Bessman, M. J., Amzel, L. M. Biochemistry 2002, 41, 9279.

Gangjee A, Yu J, McGuire J J, Cody V, Galitsky N, Kisliuk R L, Queener S F. J Med Chem Oct. 19, 2000;43(21):3837-51.

Gordon R B, Keough D T, Emmerson B T. , J Inherit Metab Dis 1987;10(1):82-8.

Hara T, Kato H, Katsube Y, Oda J. Biochemistry Sep. 17, 1996;35(37):11967-74.

Ho Y, Gruhler A, Heilbut A, Bader G D, Moore L, Adams S L, Millar A, Taylor P, Bennett K, Boutilier K, Yang L, Wolting C, Donaldson I, Schandorff S, Shewnarane J, Vo M, Taggart J, Goudreault M, Muskat B, Alfarano C, Dewar D, Lin Z., Michalickova K, Willems A R, Sassi H. Nielsen P A, Rasmussen K J, Andersen J R, Johansen L E, Hansen L H, Jespersen H. Podtelejnikov A, Nielsen E, Crawford J, Poulsen V, Sorensen B D, Matthiesen J, Hendrickson R C, Gleeson F, Pawson T, Moran M F, Durocher D, Mann M, Hogue C W, Figeys D, Tyers M. Nature Jan. 10, 2002;415(6868):180-3.

Jain J, Almquist S J, Shlyakhter D, Harding M W. J Pharm Sci May 2001;90(5):625-37.

Johansson K, Ramaswamy S, Ljungcrantz C, Knecht W, Pislcur J, Munch-Petersen B, Eriksson S, Eklund H. Nat Struct Biol 2001 8: 616.

Johansson N G, Eriksson S.. Acta Biochim Pol 1996;43(1):143-60.

Jones R J, Twelves C J. Expert Rev Anticancer Ther February 2002;2(1): 13-22.

Kan J L, Moran R G. J Biol Chem Jan. 27, 1995;270(4):1823-32.

Kaye S B. Br J Cancer 1998;78 Suppl 3:1-7.

Klinov S V, Chebotareva N A, Sheiman B M, Birinberg E M, Kurganov B I. Bioorg Khim Jul. 1, 1987;13(7):908-14.

Levdikov V M, Barynin V V, Grebenko Al, Melik-Adamyan W R, Lamzin V S, Wilson K S. Structure Mar. 15, 1998;6(3):363-76.

Li C, Kappock T J, Stubbe J, Weaver T M, Ealick S E., Structure Fold Des Sep. 15, 1999;7(9): 1155-66.

Li M H, Kowk F, Chang W R, Lau C K, Zhang J P, Lo S C, Jiang T, Liang D C. J Biol Chem Sep. 15, 2000.

Mathews I I, Kappock T J, Stubbe J, Ealick S E. Structure Fold Des Nov. 15, 1999;7(11):1395-406.

Mauritz R, Peters G J, Priest D G, Assaraf Y G, Drori S, Kathmann I, Noordhuis P, Bunni M A, Rosowsky A, Schornagel J H, Pinedo H T M, Jansen G. Biochem Pharmacol Jan. 15, 2002;63(2):105-15.

Sakai Y, Furuichi M, Takahashi M, Mishima M, Iwai S, Shirakawa M, Nakabeppu Y. J Biol Chem Mar. 8, 2002;277(10):8579-87.

Sant M E, Lyons S D, Phillips L, Christopherson R I. J Biol Chem Jun. 5, 1992;267(16):11038-45.

Saravanan V, Hamilton J, Expert Opin Pharmacother July 2002;3(7):845-56.

Sawaya M R, Kraut J. Biochemistry Jan. 21, 1997;36(3):586-603.

Schoettle S L, Christopherson R I. Adv Exp Med Biol 1994;370:151-4.

Semin Oncol 1997 Antifolates in clinical development. Takimoto C H.

Sierra E E, Goldman I D., Semin Oncol April 1999;26(2 Suppl 6):11-23.

Ubbink J B, Bissbort S, Vermaak W J, Delport R. Enzyme 1990;43(2):72-9.

van Ede A E, Laan R F, Blom H J, Boers G H, Haagsma C J, Thomas C M, De Boo T M, van de Putte L B. Rheumatology (Oxford) June 2002;41(6):658-65.

Vikram Prabhu, K. Brock Chatson, Helen Lui, Garth D. Abrams, and John King, Plant Physiol. 1998 116: 137-144.

Wall M, Shim J H, Benkovic S J. Biochemistry Sep. 19, 2000;39(37):11303-11.

Wang W, Kappock T J, Stubbe J, Ealick S E. Biochemistry Nov. 10, 1998;37(45):15647-62.

Weber G, Nagai M, Natsumeda Y, Ichikawa S, Nakamura H. Eble J N, Jayaram H N, Zhen W N, Paulik E, Hoffman R, et al., Adv Enzyme Regul 1991;31:45-67.

Weber G, Prajda N. Adv Enzyme Regul 1994; 34:71-89.

Zographos S E, Oikonomakos N G, Tsitsanou K E, Leonidas D D, Chrysina E D, Skamnaki V T, Bischoff H, Goldmann S. Watson K A, Johnson L N. Structure Nov. 15, 1997;5(11):1413-25.

The practice of the present invention will employ, unless otherwise indicated, conventional techniques of molecular biology, cell biology, cell culture, microbiology and recombinant DNA, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al; U.S. Pat. No: 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymnology, Vols. 154 and 155 (Wu et al. eds.), Immunochemical Methods In Cell And Molecular Biology (Mayer and Walker, eds., Academic Press, London, 1987). The contents of all cited references (including literature references, issued patents, published patent applications as cited throughout this application) are hereby expressly incorporated by reference.

Equivalents

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

1. A method of identifying protein target(s) which interact with a chemical compound, comprising:
(a) immobilizing said chemical compound on a support;
(b) contacting said chemical compound immobilized on said support with a sample containing potential protein target(s);
(c) isolating protein target(s) which interact with said immobilized chemical compound;
(d) determining the identity of the protein target(s) isolated in (c) by mass spectrometry, thereby identifying protein target(s) of said chemical compound.

2. The method of claim 1, wherein said suport is a magnetic support.

3. The method of claim 2, wherein the sample is a cell lysate or a tissue extract.

4. The method of claim 3, wherein said cell lysate is from a primary human cell line or a tumor cell line.

5. The method of claim 3, wherein said cell lysate is enriched for proteins specifically localized to a subcellular organelle or a membrane faction.

6. The method of claim 2, wherein said chemical compound has a desirable biological effect.

7. The method of claim 6, wherein the mechanism underlying said desirable biological effect is unclear or incomplete.

8. The method of claim 7, further comprising determining said mechanism by identifying one or more protein target(s) responsible for said desired biological effect.

9. The method of claim 6, further comprising validating one or more identified protein target(s) of said chemical compound for a different desired biological effect.

10. The method of claim 6, wherein said chemical compound is a drug candidate having one or more undesirable side effect(s).

11. The method of claim 10, further comprising determining the mechanism of said side effect(s) by identifying one or more protein target(s) responsible for said side effect(s).

12. The method of claim 11, further comprising engineering said drug candidate to eliminate interaction with protein target(s) responsible for said side effect(s), without adversely affecting said desired biological effect(s).

13. The method of claim 2, wherein in step (a), the compound is synthesized on said magnetic support.

14. The method of claim 2, wherein said magnetic support is a polymeric solid support with desirable swelling properties in both organic and aqueous solvents.

15. The method of claim 2, wherein in step (a), said compound is immobilized on said magnetic support via a covalent linker.

16. The method of claim 15, wherein said linker is optimized for protein target interaction whilst minimizing undesirable nonspecific interactions.

17. The method of claim 15, wherein said linker is non-cleavable.

18. The method of claim 15, wherein said linker is photo-labile.

19. The method of claim 2, wherein in step (a), said compound is immobilized to said magnetic support via Biotin-Avidin affinity pair.

20. The method of claim 2, wherein said compound is Methotrexate (MTX).

21. The method of claim 2, wherein said magnetic support comprises a polyethylene glycol dimethylacrylamide (PEGA) copolymer.

22. The method of claim 2, wherein the mass spectrometry is tandem mass spectrometry.

23. The method of claim 2, wherein the mass spectrometry is Fourier Transform Mass Spectrometry (FTMS).

24. The method of claim 2, wherein said sample comprising a library of secondary samples, each independently obtained from a library of ADME/Tox assays.

25. The method of claim 24, wherein said secondary samples comprise a library of serum binding proteins.

26. A method of optimizing interaction between a chemical compound and protein target(s) of said chemical compound, comprising:
(a) providing a chemical compound having one or more desired biological effect(s);
(b) identifying, by the method of claim 1, protein target(s) which interact with said chemical compound, wherein one or more of said protein target(s) has known structure;
(c) designing, by computational chemistry methodology, a library of candidate chemical compounds derived from said chemical compound, taking into consideration the known structure of said target protein(s);
(d) Identifying, if any, one or more chemical compound(s) from the library of candidate chemical compounds, wherein said one or more chemical compound(s) each interacts with said protein target(s) with higher affinity than that of said chemical compound.

27. The method of claim 26, wherein step (b) is effectuated by the method of claim 2.

28. The method of claim 27, further comprising identifying and eliminating one or more undesirable chemical compounds which non-specifically interact with proteins from multiple pathways.

29. A method of identifying interacting protein(s) for one or more compounds from a library of diverse chemical compounds having unknown biological activity, comprising:
(a) providing said library of diverse chemical compounds by solid-phase synthesis which allows for cleavage of said chemical compounds from a support;
(b) obtaining an equivalent portion of the library of chemical compounds in soluble form, for use in a panel of assays;
(c) assessing selectivity of each member of the library of chemical compounds against the panel of assays;
(d) identifying one or more compounds with selective efficacy in the panel of assays;
(e) independently identifying, using the method of claim 1, protein target(s) of each of the one or more chemical compounds identified in (d).

30. The method of claim 29, wherein said support is a magnetic support, and wherein step (e) is effectuated by the method of claim 2.

31. The method of claim 30, wherein step (b) is effected by cleavage of the library of chemical compounds from said magnetic support.

32. The method of claim 30, wherein said panel of assays relate to cellular assays which are disease models.

33. The method of claim 30, wherein step (e) is effected by directly using compounds synthesized in step (a).

34. The method of claim 30, wherein the panel of assays is a panel of ADME/Tox (Absorption, Distribution, Metabolism, and Excretion/Toxicity) assays.

35. The method of claim 30, wherein the panel of assays include assessing changes in expression level of proteins.

36. The method of claim 35, wherein the changes in expression level of proteins is assessed by FTMS (Fourier Transform Mass Spectrometry).

37. A method of identifying new drug targets within a known protein target family, comprising:
(a) providing a protein target family-specific, immobilized library of diverse chemical compounds based upon a chemical compound known to interact with said family, wherein said library of chemical compounds are immobilized on a support;
(b) contacting said immobilized library of chemical compounds with a sample containing potential protein target(s);
(c) isolating protein target(s) which interact with said immobilized library of chemical compounds;
(d) determining the identity of, if any, new protein target(s) isolated in (c) by mass spectrometry, thereby identifying new drug target(s) within said known protein target family.

38. The method of claim 37, wherein said support is a magnetic support.

39. A method of conducting a pharmaceutical business, comprising:
(i) by the method of claim 1, identifying one or more interacting protein(s) of a chemical compound with known biological effects;
(ii) validating the interacting protein(s) identified in step (i) as druggable disease targets, wherein the protein(s) were previously not known to be associated with diseases;
(iii) formulating a pharmaceutical preparation including the chemical compounds for treatment of diseases associated with the protein target(s) identified in step (ii) as having an acceptable therapeutic profile.

40. The method of claim 39, wherein step (i) is effectuated by claim 2.

41. The method of claim 40, including an additional step of establishing a distribution system for distributing the pharmaceutical preparation for sale, and may optionally include establishing a sales group for marketing the pharmaceutical preparation.

42. A method of conducting a pharmaceutical business, comprising:
(i) by the method of claim 1, identifying one or more interacting protein(s) of a compound with known biological effects;
(ii) licensing, to a third party, the rights for further drug development or target validation of the protein(s) identified in step (i).

43. The method of claim 41, wherein step (i) is effectuated by claim 2.
